(19) United States
(12) Patent Application Publication
(10) Pub. No.: US 2026/0118362 A1
(43) Pub. Date: Apr. 30, 2026

(54) METHODS AND SYSTEMS FOR IDENTIFYING HEMATOPOIETIC STEM CELL TRANSPLANT DONORS FROM AN IMMUNE SIGNATURE

(71) Applicant: Melio Healthcare Limited, London (GB)

(72) Inventors: Adam Gregory LAING, London (GB); Thomas Stewart HAYDAY, London (GB); Jeremy Carter MASON, Cambridge (GB); Eduardo DE PAIVA ALVES, London (GB); Hsiu Mien YANG, London (GB); Duncan Robert MCKENZIE, London (GB)

(73) Assignee: Melio Healthcare Limited, London (GB)

(21) Appl. No.: 19/368,649

(22) Filed: Oct. 24, 2025

Related U.S. Application Data
(60) Provisional application No. 63/712,272, filed on Oct. 25, 2024.

(51) Int. Cl. G01N 33/58; G01N 15/01; G01N 15/10; G01N 15/1434; G01N 15/149; G06N 20/00

(52) U.S. Cl. CPC G01N 33/582; G01N 15/01; G01N 15/1434; G01N 15/149; G06N 20/00; G01N 2015/1006

(57) ABSTRACT

Method and systems for sorting potential hematopoietic stem cell transplant (HSCT) donors as a donor or non-donor using immunophenotyping of blood samples. The methods and systems can be used to identify a donor for a HSCT or to choose a donor to generate a HSCT blood product that is not likely to result in bad outcomes for a recipient. Also provided herein are methods and systems for training machine learning models that can be used in methods and systems for sorting potential HSCT donors.

### CROSS-REFERENCE TO RELATED APPLICATIONS

This application claims the priority benefit of U.S. Provisional Patent Application No. 63/712,272, filed Oct. 25, 2024, the entire contents of which are incorporated herein by reference.

### FIELD

This disclosure relates to choosing hematopoietic stem cell transplant donors from immune signatures of the donor individual.

### BACKGROUND

Hematopoietic stem cell transplants (HSCT), commonly referred to as bone marrow transplants, represent a therapeutic intervention for various medical conditions, primarily involving the hematologic and immune systems. These conditions are categorized as malignant, non-malignant, or genetic disorders, with certain patient populations demonstrating a particular need for this procedure.

Selecting an appropriate donor for HSCT is crucial to maximizing the success of the procedure and minimizing complications. Such complications may be graft rejection, graft-versus-host disease (GvHD), and disease relapse. Traditionally, donors have been matched with recipients based on genetic relatedness, age, sex, and blood type, with limited success. Nowadays, donors and recipients can be matched based on their human leukocyte antigen (HLA) types. However, even with optimal HLA markers and other characteristics matching, HSCT failures still occur 20%-50% of the time, due to rejection of the graft or engraftment failures. Despite the advancements in donor selection and transplant protocols, HSCT failure remains a multifactorial issue with significant associated risks and outcomes that cannot be accurately predicted with a high degree of success. Therefore, there remains a strong need for new methods for donor selection for HSCT that allows for maximizing success and minimizing complications.

### SUMMARY

By optimizing the choice of donors using advanced immunophenotyping as provided in the present invention, choice of donors likely to contribute to successful engraftment of the HSC is improved without increased GvHD, and in some aspects, relapse in cancer treatment for the intended recipients may be reduced. Optimizing donor choice based on the whether they will respond to mobilization treatments used in preparation for HSCT donation will also improve donor success and prevent donors from expensive and potentially difficult mobilization treatments.

Provided herein are methods and systems that can be used to increase the selection of donors likely to contribute to successful engraftment of HSCs and donors likely to respond well to treatment with mobilization agents. The methods and systems described herein comprise the use machine learning models, trained using donor immunophenotyping data and recipient HSCT outcomes, to predict the probability that a potential donor will contribute to a successful donation. The probability is used to sort the potential donor as a donor (e.g. universal donor) or a non-donor. The methods comprise machine learning models trained using one or more outcomes, such as but not limited to recipient survival, disease relapse, or infection. The methods comprise machine learning models trained samples collected before and after treating a donor with a mobilization agent. Also provided herein are methods and system for training a machine learning model to predict the probability of an outcome based on immunophenotyping data from a potential HSCT donor and methods and systems for training a machine learning model to predict the response to a mobilization agent for a potential HSCT donor.

Provided herein are methods for sorting a potential Hematopoietic Stem Cell Transplantation (HSCT) donor as a donor or non-donor, the methods comprising: fluorescently labeling cells contained within a sample from a potential HSCT donor, by contacting at least an aliquot of the sample with at least one immunophenotyping fluorescent labeling panel, generating fluorescent intensity data by processing the fluorescently-labeled cells from the sample using a flow cytometer; providing at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals; outputting a predicted probability of a positive first outcome following a HSCT for the potential HSCT donor; and sorting the potential HSCT donor as a donor or a non-donor based at least on the predicted probability of a positive first outcome.

In some aspects, the potential donor is sorted as a donor if the predicted probability from the first machine learning model is greater than a predetermined threshold.

In some aspects, the methods further comprise generating a HSCT blood donation product from the potential HSCT donor if the potential HSCT donor is sorted as a donor. In some aspects, the methods further comprise treating the potential donor with a mobilization agent to mobilize hematopoietic stem cells from bone marrow to peripheral blood if the potential HSCT donor is a donor. In some aspects, the mobilization agent is granulocyte-colony stimulating factor (G-CSF).

In some aspects, the methods further comprise matching the potential HSCT donor to a recipient in need of a HSCT donation if the potential HSCT donor is sorted as a donor. In some aspects, the methods further comprise transplanting cells the from potential HSCT donor to the recipient in need of a HSCT donation.

In some aspects, the first machine learning model has been trained with results of a second machine learning model. In some aspects, the second machine learning model has been trained to predict the probability of a second outcome in a HSCT recipient with at least: (a) fluorescent intensity data, or data derived therefrom obtained from a plurality of fluorescently labeled cells from a plurality of HSCT donor individuals and (b) one or more indications of a second outcome for a plurality of recipient individuals who have each received a HSCT from an individual in the plurality of donor individuals. In some aspects, the second outcome is a clinical indication that partially explains the first outcome. In some aspects, the second outcome is a clinical indication that does not explain the first outcome. In some aspects, the second outcome is an indication of the positive first outcome collected at a second time point. In some aspects, training the first machine learning model with a results of the second machine learning model improves predictive performance of the first machine learning model.

In some aspects, the methods further comprise generating the results of a second machine learning model by providing at least a subset of the fluorescent intensity data, or data derived therefrom as input to the second machine learning model.

In some aspects, the second machine learning model is a mobilization model. In some aspects, the mobilization model is a machine learning model trained to predict donor response to a mobilization agent. In some aspects, the mobilization model has been trained to predict donor response to a mobilization agent using at least (a) a subset of pre-mobilized fluorescent intensity data, or data derived therefrom, for a plurality of donor individuals, and (b) one or more indications of the response. In some aspects, the pre-mobilized fluorescent intensity data has been generated from a plurality of fluorescently labeled cells from a pre-mobilized sample collected from a donor of a plurality of donor individuals. In some aspects, the one or more indications of the response has been generated using post-mobilized fluorescent intensity data, generated from a plurality of fluorescently labeled cells from a post-mobilized sample collected from the donor.

In some aspects, the first machine learning model has been trained with two or more indications of the positive first outcome collected at two or more timepoints. In some aspects, the predicted probability of a positive first outcome comprises the predicted probability of the positive first outcome at each of the two or more time points.

In some aspects, the sorting is based on a predicted probability of a negative first outcome outputted from a third machine learning model. In some aspects, the third machine learning model has been trained to predict the probability of a negative first outcome following a HSCT with at least (a) fluorescent intensity data, or data derived therefrom obtained from a plurality of fluorescently labeled cells from a plurality of HSCT donor individuals and (b) one or more indications of a negative first outcome for a plurality of recipient individuals who have each received a HSCT from an individual in the plurality of donor individuals.

In some aspects, the potential HSCT donor is sorted as a non-donor if the predicted probability of a negative first outcome following a HSCT is greater than a first predetermined threshold. In some aspects, the potential HSCT donor is sorted as a donor if the predicted probability of a negative first outcome following a HSCT is less than the first predeterminer threshold and the predicted probability of a positive first outcome following a HSCT is greater than a second predetermined threshold. In some aspects, additional clinical metrics are used to sort the potential HSCT donor as a donor or a non-donor if the predicted probability of a negative first outcome following a HSCT is less than the first predetermined threshold and the predicted probability of a positive first outcome following a HSCT is less than the second predetermined threshold.

In some aspects, the third machine learning model has been trained with two or more indications of the negative first outcome collected at two or more timepoints. In some aspects, the predicted probability of a negative first outcome comprises the predicted probability of the negative first outcome at each of the two or more timepoints.

In some aspects, the methods comprise generating a predicted probability of a negative first outcome by providing at least a subset of the fluorescent intensity data, or data derived therefrom as input to the third machine learning model and outputting the predicted probability of a negative first outcome.

In some aspects, the sample comprises peripheral blood cells. In some aspects, the sample comprises isolated peripheral blood mononuclear cells (PBMCs). In some aspects, the sample comprises a pre-mobilized sample. In some aspects, wherein the sample comprises a post-mobilized sample. In some aspects, the potential HSCT donor is healthy.

In some aspects, the positive first outcome is survival, lack of infection, lack of disease relapse, or lack of graft vs host disease (GvHD) between the HSCT and a defined timeframe. In some aspects, the second outcome is survival, lack of infection, lack of disease relapse, or lack of graft vs host disease (GvHD) between the HSCT and the defined timeframe. In some aspects, the negative first outcome is death when the positive first outcome is survival. In some aspects, the negative first outcome is infection when the positive first outcome is lack of infection. In some aspects, the negative first outcome is disease relapse when the positive first outcome is lack of disease relapse. In some aspects, the negative first outcome is GvHD when the positive first outcome is lack of GvHD. In some aspects, the defined timeframe is 6 months, 1 year, 2 years, 3 years, 4 years, or 5 years.

In some aspects, the first machine learning model comprises a decision tree based classification model. In some aspects, the first machine learning model comprises a XGBoost model. In some aspects, the first machine learning model comprises a logistic regression model. In some embodiments, the first machine learning model comprises an Adaptive Best Subset Selection Ensemble (ABSSE) model.

In some aspects, the second machine learning model comprises a decision tree based classification model. In some aspects, the second machine learning model comprises a XGBoost model. In some aspects, the second machine learning model comprises a logistic regression model. In some embodiments, the second machine learning model comprises an Adaptive Best Subset Selection Ensemble (ABSSE) model.

In some aspects, the third machine learning model comprises a decision tree based classification model. In some aspects, the third machine learning model comprises a XGBoost model. In some aspects, the third machine learning model comprises a logistic regression model. In some embodiments, the third machine learning model comprises an Adaptive Best Subset Selection Ensemble (ABSSE) model.

Also provided herein are methods of training a machine learning model to predict the probability of an outcome following a HSCT, the method comprising: obtaining fluorescent intensity data, generated from a plurality of fluorescently labeled cells from a donor of a plurality of donor individuals who have donated hematopoietic stem cells to a recipient individual of a plurality of recipient individuals; obtaining one or more indications of the outcome of a matched recipient individual following an HSCT from a donor in the plurality of donor individuals; and training a machine learning model to predict the probability of an outcome following a HSCT, wherein the training is based at least on (a) a subset of the fluorescent intensity data, or data derived therefrom, for the plurality of donor individuals, and (b) one or more indications of the outcome of a matched recipient following an HSCT from the plurality of donor individuals.

In some aspects, the outcome is selected from a group consisting of survival, death, disease relapse, lack of disease relapse, infection, lack of infection, lack of HvGD, or HvGD.

In some aspects, the machine learning model is further trained using two or more indication of the outcome collected at two or more timepoints. In some aspects, the predicted probability of an outcome comprises the predicted probability of the outcome at each of the two or more time points.

Also provided herein are methods of training a machine learning model to predict donor response to mobilization agents, the methods comprising: obtaining pre-mobilized fluorescent intensity data, generated from a plurality of fluorescently labeled cells from a pre-mobilized sample collected from a donor of a plurality of donor individuals; obtaining post-mobilized fluorescent intensity data, generated from a plurality of fluorescently labeled cells from a post-mobilized sample collected from the donor; obtaining one or more indications of the response of the donor to a mobilization agent from the post-mobilized fluorescent intensity data; and training a machine learning model to predict donor response to a mobilization agent, wherein the training is based at least on (a) a subset of the pre-mobilized fluorescent intensity data, or data derived therefrom, for the plurality of donor individuals, and (b) one or more indications of the response.

In some aspects, the machine learning model is further trained using information about the cellular composition of the post-mobilized sample. In some aspects, the one or more indications of the response comprise number of CD34+ cells.

In some aspects, the machine learning model comprises a decision tree based classification model. In some aspects, the machine learning model comprises a XGBoost model. In some aspects, the machine learning model comprises a Adaptive Best Subset Selection Ensemble (ABSSE) model. In some aspects, training the ABSSE model comprises multilayer feature selection. In some aspects, training the machine learning model comprises selecting two or more feature sets, training two or more machine learning model with the two or more feature sets.

In some aspects, training comprises optimizing performance using hyperparameter tuning. In some aspects, the hyperparameter tuning is performed using Grid Search. In some aspects, the hyperparameters used for hyperparameter tuning comprise, number of trees, depth of trees, a learning rate, a fraction of training samples to build each tree, and a fraction of features used by each tree controlled by overfitting parameters. In some aspects, the machine learning model comprises a logistic regression model.

In some aspects, the fluorescent intensity data, generated from a plurality of fluorescently labeled cells is generated by a method comprising; fluorescently labeling cells contained within a sample from the donor, by contacting at least an aliquot of the sample with at least one immunophenotyping fluorescent labeling panel; generating fluorescent intensity data by processing the fluorescently-labeled cells from the sample using a flow cytometer.

In some aspects, one of the at least one immunophenotyping fluorescent labeling panel comprises a panel of fluorescent-labeled antibodies directed to cell surface proteins associated with antigen-presenting cells (APCs). In some aspects, the panel of fluorescently-labeled antibodies comprises fluorescently-labeled antibodies directed to CD3, CD4, CD8, CD25, CD45, CD19, CD27, IgD, IgM, CD56, CD16, CD14, HLA-DR, CD11c, CD56, TCRgd, TCR Vα7.2. TCR Vδ1, TCR Vδ2, TCR Vα24-Jα18, CCR10, CD103/ITGAE, CD122/IL2RB, CD161/KLRB1, CD223/LAG-3, CD274/PD-L1, CD335/NKp46, CD43, CD10, CD138, CD141, CD183/CXCR3, CD185/CXCR5, CD194/CCR4, CD197/CCR7, CD279/PD-1, CD28, CD294/CRTH2, CD337/NKp30, CD38, CD39, CD5, CD62L, CD86, CD95, ICOS, TIGIT, TIM-3, CD40, KLRG1, CD69, CD196/CCR6, CD1c, CD24, CD267/TACI, CD303/BDCA-2/CLEC4C, CD31, CD319, CD57, CD127, CD45RO, CD45RA, or any combination thereof. In some aspects, the panel of fluorescently labeled antibodies comprise fluorescently-labeled antibodies directed to cell surface markers that are indicative of live cells, dead cells, or both.

In some aspects, the fluorescent intensity data, or data derived therefrom, comprises mean fluorescent intensity (MFI) data. In some aspects, the fluorescent intensity data, or data derived therefrom, comprises cell classifications. In some aspects, the cell classifications are generated from fluorescent intensity data, or data derived therefrom, comprising mean fluorescent intensity (MFI) data. In some aspects, wherein the cell classifications are generated by a cell classification machine learning model.

In some aspects, the cell classifications comprise cell ownership into at least 10, at least 20, at least 30, at least 40, at least 50, at least 60, at least 70, at least 80, at least 90, or at least 100 cell populations. In some aspects, the cell populations comprise distinct immune cell subpopulations. In some aspects, the distinct immune cell subpopulations comprise white blood cells (WBC), Eosinophils, Eosinophil/CD5+, Neutrophils, Neutrophils/big, Neutrophils/CD5+, Neutrophils/small. B-cells, B-cells/CD5-CD27−, Monocytes/CD56+, Monocytes/CD56−, NK-cells, Dendritic cells (DC), T-cells, iNKT cells, gamma delta T-cells (total GD), Vd1 cells, Vd2 cells, Vdx cells, Mucosal-associated invariant T (MAIT) cells, TEMRA cells, CD4 naïve cells, T helper cells, CD4 effector memory cells, Treg cells, Leukocytes, Helper T cells, Non-T/Non-NK B cells, Naïve T cells, Memory T cells, Naïve Cytotoxic T cells, Memory Cytotoxic T cells, Granulocytes, Activated T cells, Non-T/Non-B/Non-NK activated cells, or any combination thereof.

In some aspects, the flow cytometer is configured for at least about 5, at least about 10, at least about 15, at least about 20, at least about 30, at least about 40, at least about 50, at least about 60, at least about 70, at least about 80, at least about 90, or at least about 100 fluorescence detection channels. In some aspects, the flow cytometer is a full spectrum flow cytometer. In some aspects, the flow cytometer outputs cell classification. In some aspects, the flow cytometer outputs mean fluorescent intensity (MFI) data.

Also provided herein are systems comprising: one or more processors; an input device; a memory communicative coupled to the one or more processors and the input device and configured to store instructions that, when executed by the one or more processes, cause the system to: receive user defined threshold, receive fluorescent intensity data generated by processing fluorescently-labeled sells from a sample from a potential HSCT donor using a flow cytometer; provide at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals; output a predicted probability of a positive first outcome following a HSCT for the potential HSCT donor; and sort the potential HSCT donor as a donor or a non-donor based at least on the predicted probability of a positive first outcome, wherein the potential donor is sorted as a donor if the predicted probability from the first machine learning model is greater than the user defined threshold.

Also provided herein are systems comprising, one or more processors; a memory communicative coupled to the one or more processors and configured to store instructions that, when executed by the one or more processes, cause the system to: obtain fluorescent intensity data, generated from a plurality of fluorescently labeled cells from a donor of a plurality of donor individuals who have donated hematopoietic stem cells to a recipient individual of a plurality of recipient individuals; obtain one or more indications of the outcome of a matched recipient individual following an HSCT from a donor in the plurality of donor individuals; and train a machine learning model to predict the probability of an outcome following a HSCT, wherein the training is based at least on (a) a subset of the fluorescent intensity data, or data derived therefrom, for the plurality of donor individuals, and (b) one or more indications of the outcome of a matched recipient following an HSCT from the plurality of donor individuals.

Also provided herein are systems comprising: one or more processors; a memory communicative coupled to the one or more processors and configured to store instructions that, when executed by the one or more processes, cause the system to: obtain pre-mobilized fluorescent intensity data, generated from a plurality of fluorescently labeled cells from a pre-mobilized sample collected from a donor of a plurality of donor individuals; obtain post-mobilized fluorescent intensity data, generated from a plurality of fluorescently labeled cells from a post-mobilized sample collected from the donor; obtain one or more indications of the response of the donor to a mobilization agent from the post-mobilized fluorescent intensity data; and train a machine learning model to predict donor response to a mobilization agent, wherein the training is based at least on (a) a subset of the pre-mobilized fluorescent intensity data, or data derived therefrom, for the plurality of donor individuals, and (b) one or more indications of the response.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawing of which:

FIG. 1 provides a non-limiting example of a method for sorting potential HSCT donors as a donor or non-donor according to some embodiments described herein.

FIG. 2 provides a non-limiting example of a method for sorting potential HSCT donors as a donor or non-donor according to some embodiments described herein.

FIG. 3 provides a non-limiting example of a method for sorting potential HSCT donors as a donor or non-donor according to some embodiments described herein.

FIG. 4 provides a non-limiting example of a method for sorting potential HSCT donors as a donor or non-donor according to some embodiments described herein.

FIG. 5 provides a non-limiting example for training a machine learning model to predict the probability of an outcome following a HSCT according to some embodiments described herein.

FIG. 6 provides a non-limiting example for training a machine learning model to predict donor response to a mobilization agent according to some embodiments described herein.

FIG. 7 shows an exemplary process for Adaptive Best Subset Selection Ensemble (ABSSE) modeling according to some of the embodiments described herein.

FIG. 8 illustrates an exemplary computing system, in accordance with some of the embodiments and systems described herein.

FIG. 9 illustrates features selected by a RandomForest model trained with an exemplary data set according to their importance for predicting survival following an HSCT.

FIG. 10 illustrates features selected by a RandomForest model trained with an exemplary data set according to their importance for predicting disease relapse following an HSCT.

FIG. 11 illustrates features selected by a RandomForest model trained with an exemplary data set according to their importance for predicting survival following an HSCT.

FIG. 12 illustrates features selected by a RandomForest model trained with an exemplary data set according to their importance for predicting survival following an HSCT using predicted disease relapse probability as a feature.

FIG. 13 illustrates features selected by a RandomForest model trained with an exemplary data set according to their importance for predicting survival following an HSCT.

FIG. 14A-14C illustrate performance of an ABSSE model predicting clinical relapse of AML patients following hematopoietic stem cell transplantation based upon the immune profile of the donor. FIG. 14A shows performance as measured by a confusion matrix and balanced accuracy of relapse classification. FIG. 14B shows performance as measured by a ROC-AUC curve. FIG. 14C shows relapse incidence estimation over time using the ABSSE model, a log rank test was used to generate a p-value comparing the no-relapse and relapse groups.

### DETAILED DESCRIPTION

Provided herein are methods and systems that can be used to increase the selection of donors likely to contribute to successful engraftment of HSCs. The methods rely in part on the insight that complex immunophenotyping using donor blood cells pre or post mobilization can inform the success of a HSCT. Immunophenotyping according to the methods described herein is likely to be predictive for HSCT success because the immune profile of a donor and the immune profile of the mobilized blood product for donation plays a large role in transplant success. The immune profile is partially considered in donor selection when HLA profiles are matched between donors and a recipients. Because transplant can fail despite a matched HLA, additional factors must be important for predicting success of a HSCT. Other components of the donor immune profile, such as specific immune cells subsets and prior exposure to pathogens likely influence HSCT success as well. Ogonek et al. Immune Reconstitution after Allogenic Hematopoietic Stem Cell Transplant, 2016, Front. Immunol. Thus, incorporating additional immunophenotyping, beyond HLA type, such as information from a detailed immune profile before and/or after mobilization improves the ability to characterized a donor as likely favorable to all recipient (e.g. universal) or for a subset of recipients (e.g. recipients suffering from certain diseases). These markers that can be identified and captured in a donor immune profile based on a pre-mobilized sample or a post-mobilized sample, include but are not limited to markers correlated with stem cell quality and reduced graft vs host disease (GvHD). In addition, characterization of immune profiles for pre-mobilized and post-mobilizes samples can inform why some donors respond better to mobilization and as a result contribute to better outcomes for recipients.

The invention also relies in part on improved methods immunophenotyping. Conventionally methods for generating an immune profile by immunophenotyping include, e.g., enzyme-linked immunosorbent assays (ELISAs), immunoblotting techniques, and flow cytometry-based techniques include the use of panels of fluorescently-labeled antibodies directed to a variety of cell surface receptors and manual gating of the flow cytometry data. These techniques are often laborious and time consuming and are not easily scalable to a level that enables the processing of hundreds or thousands of samples. Recently, high throughput manifestations of deep phenotyping methods such as full spectrum flow cytometry have been developed as cost-effective techniques for immune cell profiling. The use of immune profiling needs only a non-invasive blood test that requires only a small volume of blood, enhancing patient comfort and enabling frequent testing with reduced burden on clinical infrastructure and costs.

By utilizing immunophenotyping and flow cytometry, the methods identify detailed immune cell signatures that can then be used in training and using machine learning algorithms for predicting an outcome following a HSCT. The methods described herein can thus be used to identify donors, such as potential universal donors for HSCT. Using samples from blood banks or other large donor pools, the methods can be used to prioritize or rank potential donors by the likelihood of a successful transplant without needed to collect additional information from the donor.

The methods described herein can be used in combination with traditional methods of matching donors, such as HLA status, age, sex, and blood type to match a donor (e.g. universal donor) to a recipient. By matching donors using a pool of donors already likely to contribute to a successful donation, the chances of success for an HSCT will increase and the changes for an infection, GvHD or relapse of disease decrease.

### I. Definitions

Unless otherwise defined, all technical terms used herein have the same meaning as commonly understood by one of ordinary skill in the field to which this disclosure belongs.

As used in this specification and the appended claims, the singular forms “a”, “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

Throughout this application, various parameter values may be presented in a range format. It should be understood that the description in range format is merely for convenience and brevity, and should not be construed as an inflexible limitation on the scope of the disclosure. Accordingly, the description of a range should be considered to have specifically disclosed all possible subranges as well as individual numerical values within that range, irrespective of whether a specific numerical value or specific sub-range is expressly stated. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 1.4, 2, 3, 3.6, 4, 5, 5.8, and 6. This applies regardless of the breadth of the range.

Numbers may be expressed herein as being “about” a particular value. Similarly, ranges may be expressed herein as from “about” one particular value and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

It should be recognized that use of ordinal terms such as “first” and “second” in the description of methods and systems disclosed herein does not by itself connote any priority, order of importance of one system component over another, or the temporal order in which acts of a method are performed, but are used merely as labels to distinguish, for example, one system component having a certain name from another system component having the same name but for the use of the ordinal term to distinguish the two system components.

Additionally, various implementations of the methods and systems set forth herein may be described in terms of exemplary block diagrams, process flow charts, and other illustrations. As will become apparent to one of ordinary skill in the art after reading this document, the various implementations set forth herein can be implemented without confinement to the illustrated examples. For example, block diagrams and their accompanying description should not be construed as mandating a particular architecture or configuration. Similarly, in exemplary process flow charts, some blocks are optionally combined, the order of some blocks is optionally changed, and some blocks are optionally omitted. In some implementations, additional steps may be performed in combination with the exemplary processes. Accordingly, the methods and systems as described and illustrated in greater detail below are exemplary by nature and, as such, should not be viewed as limiting.

As used herein, the terms “flow cytometry” and “flow cytometer” refer to a technique and instrument, respectively, for performing flow cytometry where the instrument is configured to capture emission of fluorescent molecules using arrays of highly sensitive light detectors, thereby enabling the capture of highly multiplexed fluorescence intensity data sets. This includes all variants of flow cytometry and mass cytometry technology, including but not limited to conventional flow cytometry and full spectrum flow cytometry.

As used herein, the term “immunophenotyping panel” refers to a panel of binding agents, for example antibodies, abdurins, affibodies, affimers, affitins, anticalins, bicyclic peptides, darpins, fynomers, kunitz domains, and monobodies, (e.g., fluorescently-labeled antibodies) that bind to specific antigens or markers present on the surface of the cells, or in some cases, within the cell. These binding agents are labeled, such as fluorescently labeled, such that flow cytometry may be used to identify cells that have the antigen or marker for which the binding agent is specific. In one embodiment, fluorescently antibodies are used in the immunophenotyping panel.

As used herein a “donor” for hematopoietic stem cell transplantation (HSCT) refers to an individual or entity that provides hematopoietic stem cells (HSCs) for the purpose of transplantation into a recipient. Hematopoietic stem cells are multipotent cells found in bone marrow, peripheral blood, or umbilical cord blood that have the ability to regenerate and differentiate into all types of blood cells. These cells are critical for reconstituting the recipient's blood and immune systems, often after the recipient undergoes treatments that destroy their own hematopoietic system, such as chemotherapy or radiation therapy. A donor can be allogeneic, where the donor and recipient are unrelated, autologous, where the donor and recipient are the same, and haploidentical, where the donor and recipient are related. A donor may have already provided HSCs for the purpose of transplantation into a recipient, or the donor may be eligible to provide HSCs for the purpose of transplantation into a recipient.

The section headings used herein are for organizational purposes only and are not to be construed as limiting the subject matter described. The description is presented to enable one of ordinary skill in the art to make and use the invention and is provided in the context of a patent application and its requirements.

The disclosures of all publications, patents, and patent applications referred to herein are each hereby incorporated by reference in their entireties. To the extent that any reference incorporated by reference conflicts with the instant disclosure, the instant disclosure shall control.

### II. Methods for Sorting a Potential HSCT Donor

Provided herein are methods that can be used to sort a potential HSCT donor as a donor (e.g. universal donor) or a non-donor. The methods comprise generating an immune profile for the potential donor, inputting it into a machine learning model trained to predict the probability of a positive outcome (e.g. survival) following a HSCT, outputting a probability of the positive outcome (e.g. recipient survival) for the potential donor, and sorting the potential donor as a donor (e.g. universal donor) or a non-donor.

A. Generating Fluorescent Intensity Data for a Sample from a Potential HSCT Donor

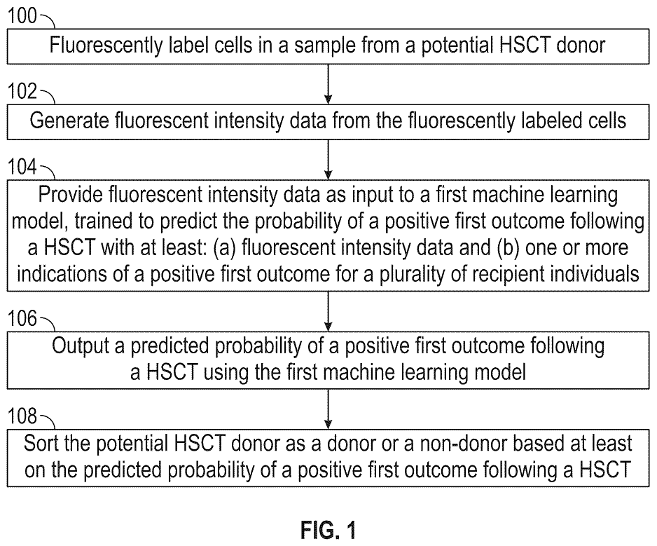

FIG. 1 provides and exemplary embodiment of a method for sorting a potential HSCT doner as a donor or non-donor as described herein. At block 101, cells contained within a sample from a potential HSCT donor are fluorescently labeled by contacting at least an aliquot of the sample with at least one immunophenotyping fluorescent labeling panel as described herein. In some embodiments, the sample is a fresh blood sample collected from the potential HSCT donor. In some embodiments the sample is a cryopreserved blood sample that was collected from the HSCT donor and preserved according to methods known in the art. In some embodiments, the sample comprises peripheral blood cells. In some embodiments, the sample comprises isolated peripheral blood mononuclear cells (PBMCs). In some embodiments, the methods comprise isolating PBMCs from a sample comprising peripheral blood samples.

In some embodiments, the sample comprises a pre-mobilized sample. A pre-mobilized sample may be a sample collected from the potential HSCT donor, wherein the potential HSCT donor has not been treated with a mobilization agent to mobilize hematopoietic stem cells from bone marrow to peripheral blood. In some embodiments, the potential HSCT donor is healthy.

In some embodiments, the sample comprises a post-mobilized sample. A post-mobilized sample may be a sample collected from the potential HSCT donor, wherein the potential HSCT donor has been treated with a mobilization agent to mobilization agent to mobilize hematopoietic stem cells from bone marrow to peripheral blood. In some embodiments, the mobilization agent is granulocyte-colony stimulating factor (G-CSF). In some embodiments, the post-mobilized sample comprises stem cells. In some embodiments, the stem cells are CD34+ cells. In some embodiments, the stem cells are hematopoietic stem cells.

In some embodiments, a potential HSCT donor is a healthy individual. In some embodiments, a potential HSCT donor is an individual who has donated a blood sample to blood bank. In some embodiments, a potential HSCT donor is an individual who has volunteered to provide a sample for a HSCT. In some embodiments, a potential HSCT donor has no contraindications for donating material for a HSCT. Contraindications may include prior treatment with immune stimulation treatments or immune suppressing agents. Chemotherapy may be the immune suppressing agent.

In some embodiments, fluorescent labeling cells contained within the sample comprises contacting at least an aliquot of the sample with at least one immunophenotyping fluorescent labeling panel as described herein. In some embodiments, the sample is separated into one or more aliquots and each aliquot is contacted with an immunophenotyping labeling panel. The one or more immunophenotyping fluorescent labeling panel may be any of the labeling panels described herein. In some embodiments, one of the at least one immunophenotyping fluorescent labeling panel comprises a panel of fluorescent-labeled antibodies directed to cell surface proteins associated with antigen-presenting cells (APCs), as described herein.

At block 102, fluorescent intensity data is generated by processing the fluorescently labeled cells from block 100 using a flow cytometer. Any of the methods described herein for generating fluorescent intensity data may be used at block 102. In some embodiments, the fluorescent intensity data may comprise mean fluorescent intensity (MFI) data, as described herein. In some embodiments, the flow cytometer outputs the MFI data. In some embodiments, the fluorescent intensity data may comprise cell classifications as described herein. In some embodiments, the flow cytometer outputs the cell classification. In some embodiments, the cell classifications comprise cell ownership in cells populations as described herein.

### B. Predicting the Probability of an Outcome Based on One or More Machine Learning Models

At block 104, at least a subset of the fluorescent intensity data generated at block 102, or data derived therefrom is provided as input to a first machine learning model. The first machine learning model has been trained to predict the positive first outcome with at least: fluorescent intensity data, or data derived therefrom obtained from a plurality of fluorescently labeled cells from a plurality of HSCT donor individuals and one or more indications of a positive first outcome for a plurality of recipient individuals who have each received a HSCT from an individual in the plurality of donor individuals. In some embodiments, the first machine learning model has been trained according to any of the methods described herein. In some embodiments, the methods training the first machine learning model used in block 104 using the methods described herein.

In some embodiments, the positive first outcome is a first outcome representing a positive result for a recipient who received the HSCT. In some embodiments, the first outcome is any of the outcomes described herein. In some embodiments, the positive first outcome is survival, lack of infection, lack of disease relapse, lack of graft vs host disease (GvHD), and/or evidence of graft vs tumor effect. In some embodiments, the positive first outcome is measured in a defined timeframe. The defined timeframe may be any of the defined timeframes as described herein. In some embodiments, the positive first outcome is survival between the HSCT and one year.

In some embodiments, the first machine learning models is a machine learning model trained with fluorescent intensity data, or data derived therefrom obtained from a plurality of fluorescently labeled cells from a plurality of HSCT donor individuals who have similar characteristics as the potential HSCT donor. It is contemplated that one or more first machine learning model can be trained with populations of HSCT donor individuals who have varying characteristic to improve the performance of the first machine learning model when the characteristics of the HSCT donor individuals match the potential HSCT donor. The characteristics may be characteristics known in the art to affect success of a HSCT. In some embodiments, the characteristics comprise one or more of sex, an age range, an HLA type, health status, or a weight range. In some embodiments, the characteristics comprise a characteristic of the one or more recipient who received the HSCT with a product derived from the donor, such as but not limited to the disease indication the transplant was intending to treat. In some embodiments, the methods disclosed herein comprise selecting a first machine learning model according to the characteristics of the potential HSCT donor and the training data used to train the first machine learning model.

In some embodiments, the first machine learning model has been trained with two or more indication of the positive first outcome collected at two or more timepoints. In some embodiments, the two or more timepoints are daily, monthly, or yearly. In some embodiments, the two or more timepoint are any timepoints within a defined timeframe, such as any defined timeframe describer herein.

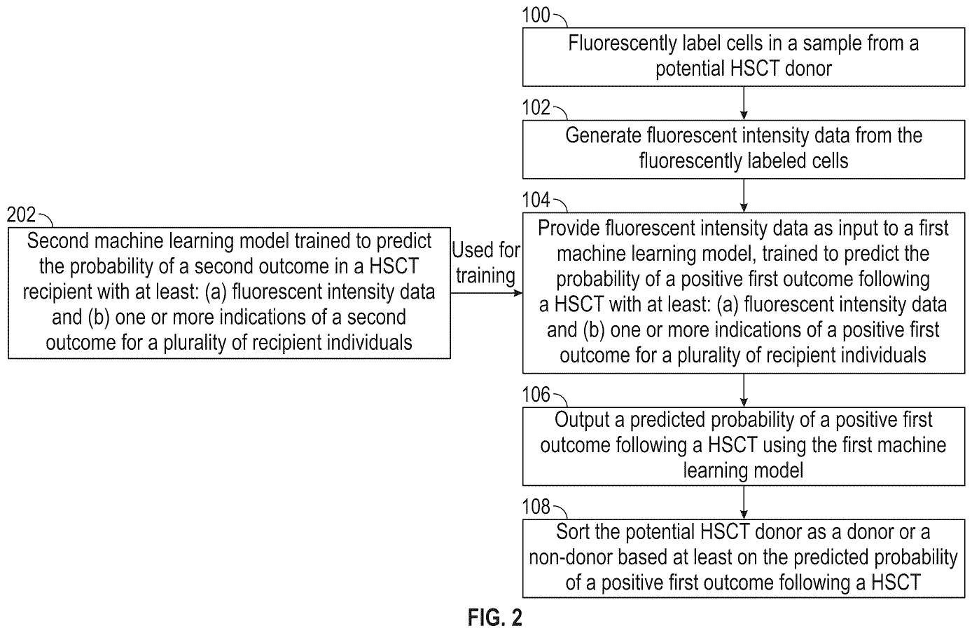

In some embodiments, the first machine learning model at block 106 has been trained with the results of a second machine learning model according to FIG. 2. In some embodiments, at block 104, a second machine learning model as exemplified at block 202 has been used to train the first machine learning model at block 104.

In some moments, the second machine learning model, block 202, is a model designed according to any of the machine learning model architectures described herein. In some embodiments, the second machine learning model comprises a decision tree based classification model as described herein. In some embodiments, the second machine learning model comprises a logistic regression model as described herein.

In some embodiments, the results of the second machine learning model comprise features selected by the second machine learning model as predictive of the outcome the second machine learning model is trained to predict. In some embodiments, the outcome is a second outcome. In some embodiments, the second outcome is any outcome for a recipient of an HSCT as described herein.

In some embodiments, the second outcome is the positive first outcome collected at a second time point. In some embodiments, the second timepoint is any timepoint during a predefined timeframe, as described herein.

In some embodiments, the second machine learning model, block 202, has been trained to predict the probability of a second outcome in a HSCT recipient with at least: (a) fluorescent intensity data, or data derived therefrom obtained from a plurality of fluorescently labeled cells from a plurality of HSCT donor individuals and (b) one or more indications of a second outcome for a plurality of recipient individuals who have each received a HSCT from an individual in the plurality of donor individuals. In some embodiments, training the first machine learning model with the results of the second machine learning model improves the predictive performance of the first machine learning model.

In some embodiments, the second outcome is a clinical indication that partially explain the positive first outcome. In some embodiments, the positive first outcome is survival, and the second outcome is disease relapse. Accordingly, the first machine learning model may benefit from training with the results of the second machine learning model because disease relapse partially explains survival. In some embodiments, the clinical indication is any of the outcomes described herein or another clinical characteristic of the plurality or recipient individuals who have received an HSCT from an individual in the plurality of donor individuals used for training the second machine learning model. In some embodiments, the second outcome is a clinical indication that does not explain the positive first outcome.

In some embodiments, the methods comprise training the second machine learning model according to the methods described herein. In some embodiments, the methods comprise comprising generating the results of a second machine learning model by providing at least a subset of the fluorescent intensity data, or data derived therefrom as input to the second machine learning model.

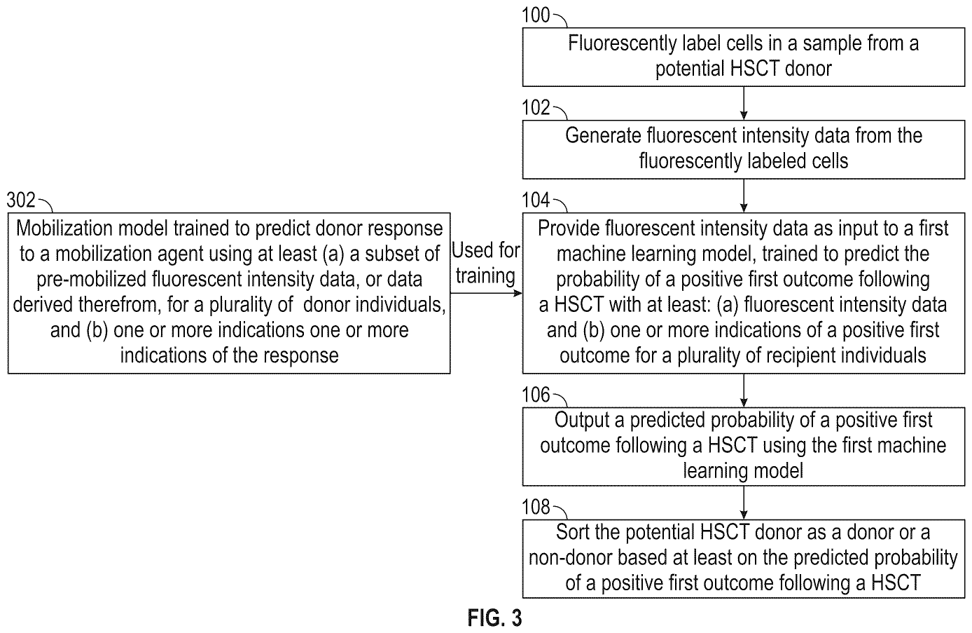

In some embodiment, the second machine learning model is a mobilization model. The incorporation of a mobilization model according to some of the embodiments disclosed herein is represented in FIG. 3. In some embodiments, at block 104, a mobilization model as exemplified at block 302 has been used to train the first machine learning model at block 104. It is appreciated that the benefits conferred by incorporation of the mobilization model emerge in embodiments of the method wherein the sample from the potential HSCT donor is a pre-mobilized sample as described herein.

In some embodiments, the results of the second machine learning model comprise features selected by the mobilization model as predictive of the one or more indications of the response of a donor to treatment with a mobilization agent. In some embodiments, the one or more indication of the response is a factor associated with a high quality HSCT product as described herein.

In some embodiments, the mobilization model is a machine learning model trained to predict donor response to a mobilization agent. The mobilization model may be trained according to any of the methods described herein for training a model to predict donor response to a mobilization agent.

In some embodiments, the mobilization model has been trained to predict donor response to a mobilization agent using at least (a) a subset of pre-mobilized fluorescent intensity data, or data derived therefrom, for a plurality of donor individuals, and (b) one or more indications of the response. In some embodiments, the model has been trained with information about the cellular composition of the post-mobilized sample as described herein.

In some embodiments, the pre-mobilized fluorescent intensity data has been generated from a plurality of fluorescently labeled cells from a pre-mobilized sample collected from a donor of a plurality of donor individuals.

In some embodiments, the one or more indications of the response has been generated using post-mobilized fluorescent intensity data, generated from a plurality of fluorescently labeled cells from a post-mobilized sample collected from the donor. In some embodiments, the one or more indications of the response has been generated using post-mobilized fluorescent intensity data and pre-mobilized fluorescent intensity data. In some embodiments, the one or more indications of the response may be any of the indication of a donor response to a mobilization agent as described herein. In some embodiments, the first machine learning model at block 106 has been trained with the results of one or more machine learning models such as a second, third, or fourth machine learning model each trained to predict one of any of the outcomes described herein, at one or more timepoints. In some embodiment's, the first machine learning models is trained with the results of all of the additional machine learning models. In some embodiments, the results of each machine learning model are used to train a successive machine learning model until the final machine learning model is the first machine learning model according to block 106.

In some embodiments, the first machine learning model at block 106 has been trained with the results of one or more machine learning models such as a second, third, or fourth machine learning model each trained to predict one of any of the outcomes described herein at one or more time points and the mobilization model. In some embodiment's, the first machine learning models is trained with the results of all of the additional machine learning models. In some embodiments, the results of each machine learning model are used to train a successive machine learning model until the final machine learning model is the first machine learning model according to block 106.

At block 106, a predicted probability of a positive first outcome following HSCT for the potential HSCT donor is outputted from block 104. In some embodiments, the predicted probability of a positive first outcome following a HSCT for the potential HSCT donor is outputted from the first machine learning model. The probability of a positive first outcome following a HSCT for the potential HSCT donor is the probability that the recipient of a HSCT from the potential HSCT donor will have the positive first outcome used as a target variable in the training of the first machine learning model. The positive first outcome may be any of the outcomes as described herein. In some embodiments, the predicted probability of a positive first outcome comprises a predicted probability of the positive first outcome at each of two or more timepoints.

In some embodiments, the first machine learning model outputs a classification result such as a classification of whether a HSCT donation from the potential HSCT donation is likely to result in the positive first outcome or not. In some embodiments, the predicted probability is the confidence the first machine learning model has in the classification. In some embodiments, the classification is used for sorting at block 108.

### C. Sorting a Donor According to the Results of One or More Machine Learning Models

At block 108, the potential HSCT donor is sorted as a donor, or a non-donor based at least on the predicted probability of a positive first outcome from block 106. In some embodiments, the donor is sorted as a universal donor or a non-universal donor. In some embodiments, a universal donor is characterized as a donor likely favorable for two or more recipients. In some embodiments, a universal donor is characterized as a donor likely favorable for any recipient with a particular disease indication. In some embodiments, a universal donor is characterized as a donor likely favorable for any recipient. In some embodiments, a favorable HSCT is an HSCT predicted to lead to one or more of the outcomes related to a successful HSCT as described herein, such as a positive first outcome.

The sorting at block 108, may also be based on characteristics traditionally associated with successful HSCT. The characteristics may include age, sex, HLA type, health, or a weight range.

In some embodiments, the predicted probability of a positive first outcome at each of two or more timepoints is used for sorting a potential HSCT donor as a donor or non-donor. In some embodiments, a potential HSCT donor is sorted as a donor if the probability of the positive first outcome remains steady or increases over time. In some embodiments, a potential HSCT donor is sorted as a donor if the probability of the positive first outcome is greater than a predetermined threshold at each of the two or more timepoints.

In some embodiments, if the potential HSCT donor is sorted as a donor, the methods may comprise any of the potential applications as described herein. In some embodiments, if the potential HSCT donor is sorted as a non-donor, the methods may comprise performing additional testing to determine if the potential HSCT donor can donate a sample for an HSCT.

In some embodiments, block 108 comprises the potential donor is sorted as a donor if the predicted probability from the first machine learning model is greater than a predetermined threshold.

In some embodiments, the sorting at block 108 is based on a predicted probability of an additional outcome from an additional machine learning model. In some embodiments, the sorting at block 108 is based on a predicted probability of a negative first outcome outputted from a third machine learning model. In some embodiments, the third machine learning model has been trained to predict the probability of a negative first outcome following a HSCT with at least (a) fluorescent intensity data, or data derived therefrom obtained from a plurality of fluorescently labeled cells from a plurality of HSCT donor individuals and (b) one or more indications of a negative first outcome for a plurality of recipient individuals who have each received a HSCT from an individual in the plurality of donor individuals.

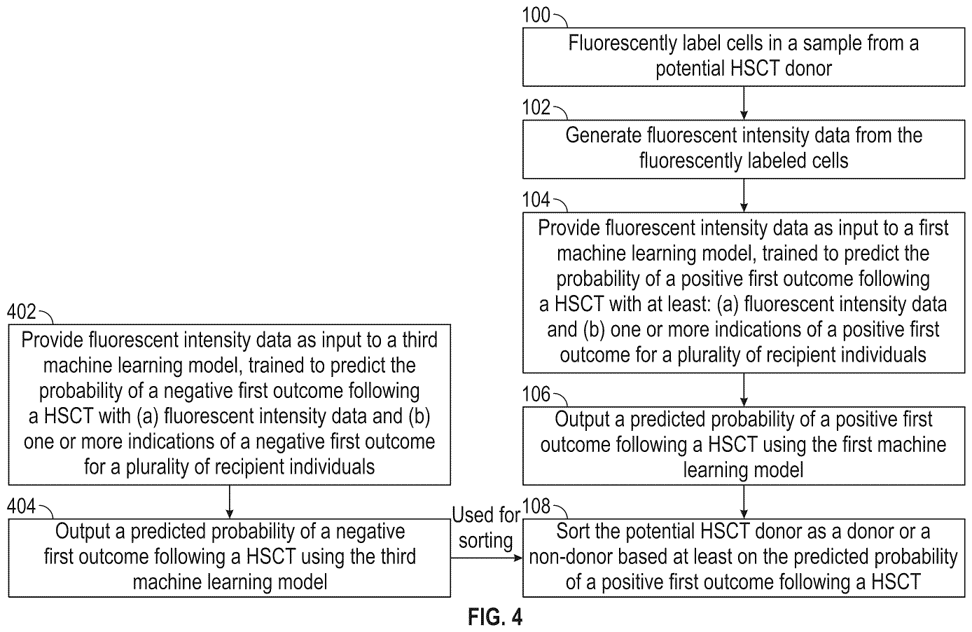

A non-limiting embodiment is displayed in FIG. 4. In some embodiments, at block 302, the fluorescent intensity data or data derived therefrom from the potential HSCT donor is inputted into a third machine learning model trained to predict the probability of a negative first outcome following a HSCT. In some embodiments, the third machine learning model has been trained with at least (a) fluorescent intensity data, or data derived therefrom obtained from a plurality of fluorescently labeled cells from a plurality of HSCT donor individuals and (b) one or more indications of a negative first outcome for a plurality of recipient individuals who have each received a HSCT from an individual in the plurality of donor individuals. In some embodiments, the predicted probability of a negative first outcome following a HSCT is outputted at block 404, using the third machine learning model at block 402. In some embodiments the fluorescent intensity data or data derived therefrom from the potential HSCT donor is the output of block 102 as described herein. In some embodiments, the predicted probability at block 404 is used to sort the potential HSCT donor as a donor or non-donor at block 108. It is appreciated that blocks 402 and 404 can be performed in parallel with blocks 104 and 106, before blocks 104 and 106 or after blocks 104 and 106.

In some embodiments, the third machine learning model has been trained with two or more indication of the negative first outcomes collected at two or more timepoints. In some embodiments, the predicted probability of a negative first outcome comprises the predicted probability of the negative first outcome at each of the two or more timepoints.

It is contemplated that predicting a probability of both a positive first outcome and a negative first outcome may improve the ability of the methods to sort potential HSCT donors. Although the data used to train the models may mirror each other in some respects, the selected features may differ. It is also contemplated that a model trained to predict positive outcomes and a model trained to predict negative outcomes may be more or less accurate at different tails of a predictive distribution than the other model. Accordingly, by using the output from both, the method may take advantage of the precision in each of the models even if the user does not know which tail the potential donor is likely to fall into on a distribution of potential positive or negative outcomes.

In some embodiments, the negative first outcome is an outcome related to the positive first outcome as described herein. The negative outcome may be an outcome related to an unsuccessful HSCT as described herein. In some embodiments, the negative first outcome is an outcome at anytime between the HSCT and a defined timeframe as described herein. In some embodiments, the negative first outcome is a negative outcome within the first year of the HSCT.

In some embodiments, the negative first outcome is death when the positive first outcome is survival. In some embodiments, the negative first outcome is death when the positive first outcome is survival. In some embodiments, the negative first outcome is infection when the positive first outcome is lack of infection. In some embodiments, the negative first outcome is disease relapse when the positive first outcome is lack of disease relapse. In some embodiments, the negative first outcome is GvHD when the positive first outcome is lack of GvHD.

In some embodiments, the sorting is based on a predicted probability of a negative first outcome outputted from a third machine learning model. In some embodiments, the potential HSCT donor is sorted as a non-donor if the predicted probability of a negative first outcome following a HSCT is greater than the first predeterminer threshold as described herein.

In some embodiments, both the probability of a positive first outcome and the probability of a negative first outcome are used to sort the potential HSCT donor as a donor or non-donor. In some embodiments, the potential HSCT donor is sorted as a donor if the predicted probability of a negative first outcome following a HSCT is less than the first predetermined threshold and the predicted probability of a positive first outcome following a HSCT is greater than a second predetermined threshold.

In some embodiments, the predicted probability of a positive first outcome at two or more timepoints and/or the predicted probability of a negative first outcome at two or more timepoints are used to sort the potential HSCT donor as a donor or a non-donor. In some embodiments, the potential HSCT donor is sorted as a non-donor if the predicted probability of a negative first outcome is higher at earlier time points. In some embodiments, the potential HSCT donor is sorted as a donor if the predicted probability of a positive first outcome is higher at all timepoints.

In some embodiments, the highest predicted probability for a positive first outcome is selected and used as the probability of a positive first outcome for the sorting methods described herein. In some embodiments, the mean predicted probability for a positive first outcome is selected and used as the probability of a positive first outcome for the sorting methods described herein.

In some embodiments, the highest predicted probability for a negative first outcome is selected and used as the probability of a positive first outcome for the sorting methods described herein. In some embodiments, the mean predicted probability for a negative first outcome is selected and used as the probability of a positive first outcome for the sorting methods described herein.

In some embodiments, additional clinical metrics are used to sort the potential HSCT donor as a donor or a non-donor if the predicted probability of a negative first outcome following a HSCT is less than the first predetermined threshold and the predicted probability of a positive first outcome following a HSCT is less than the second predetermined threshold. In some embodiments, the additional clinical metrics are characteristics traditionally associated with successful HSCT. The characteristics may include age, sex, HLA type, health, or a weight range.

In some embodiments, the first predetermined threshold is any of the predetermined thresholds as described herein. In some embodiments, the second predetermined threshold is any of the predetermined thresholds as described herein. In some embodiments, the first and second predetermined thresholds are the same. In some embodiments, the first and second predetermined thresholds are different according to any of the factors described herein for determining a threshold.

In some embodiments, the predetermined threshold is between about 20% and about 100%. In some embodiments, the predetermined threshold is between 20% and 95%, between 20% and 90%, between 20% and 85%, between 20% and 80%, between 20% and 75%, between 20% and 70%, between 20% and 65%, between 20% and 60%, between 20% and 55%, between 20% and 50%, between 20% and 45%, between 20% and 40%, between 20% and 35%, between 20% and 30%, or between 20% and 25%. In some embodiments, the predetermined threshold is between 25% and 100%, between 30% and 100%, between 35% and 100%, between 40% and 100%, between 45% and 100%, between 50% and 100%, between 55% and 100%, between 60% and 100%, between 65% and 100%, between 70% and 100%, between 75% and 100%, between 80% and 100%, between 90% and 100%, or between 95% and 100%. In some embodiments, the predetermined threshold is about 20%, 25%, 30%, 35%, 40%, 45%, 50%, 60%, 65%, 70%, 75%, 80%, 85%, 90%, 95% or 100%.

In some embodiments, the methods comprise performing the steps at blocks 102-106, 202, 302, and/or 402-404 for a plurality of potential HSCT donors. In some embodiments, the sorting at block 108 comprises ranking the plurality of potential HSCT donors. In some embodiments, a potential HSCT donor is sorted as a donor or non-donor based on their position in the ranked list of the plurality of potential HSCT donors. In some embodiments, a potential HSCT donor is sorted as a donor if they fall at the top of the distribution for predicted probability of a positive first outcome. The top of the distribution may comprise 20%, 25%, 30%, 35%, 40%, 45%, 50%, 60%, 65%, 70%, 75%, 80%, 85%, 90%, or 95% of potential HSCT donors. The top of the distribution may comprise potential HSCT donors above the first or second standard deviation above the mean for potential HSCT donors.

In some embodiments, a potential HSCT donor is sorted as a non-donor if they fall at the bottom of the distribution for predicted probability of a positive first outcome. The bottom of the distribution may comprise 5%, 10%, 15%, 20%, 25%, 30%, 35%, 40%, 45%, 50%, 60%, 65%, 70%, 75%, or 80% of potential HSCT donors. The bottom of the distribution may comprise potential HSCT donors below the first or second standard deviation below the mean for potential HSCT donors.

In some embodiments, a potential HSCT donor is sorted as a donor if they fall at the bottom of the distribution for predicted probability of a negative first outcome. The bottom of the distribution may comprise 5%, 10%, 15%, 20%, 25%, 30%, 35%, 40%, 45%, 50%, 60%, 65%, 70%, 75%, or 80% of potential HSCT donors. The bottom of the distribution may comprise potential HSCT donors below the first or second standard deviation below the mean for potential HSCT donors.

In some embodiments, a potential HSCT donor is sorted as a non-donor if they fall at the top of the distribution for predicted probability of a negative first outcome. The top of the distribution may comprise 20%, 25%, 30%, 35%, 40%, 45%, 50%, 60%, 65%, 70%, 75%, 80%, 85%, 90%, or 95% of potential HSCT donors. The top of the distribution may comprise potential HSCT donors above the first or second standard deviation above the mean for potential HSCT donors.

In some embodiments, a potential HSCT donor is sorted as a donor or a non-donor based on where they fall in the distribution for predicted probability of a positive first outcome and the distribution for predicted probability of a negative first outcome. In some embodiments, a potential HSCT donor is sorted as a donor if they fall closer to the top of the distribution for a positive first outcome than negative first outcome.

It is contemplated that the predetermined thresholds and the relationships between one or more predetermined thresholds may vary based on the use of the methods described herein. The predetermined threshold may vary based on the outcome the predicted probability is related to. For example, a predetermined threshold for the probability a recipient survives a HSCT may be higher than a predetermined threshold for the probability a recipient has an infection following an HSCT. This may be because an infection is treatable, and the physician can monitor the recipient for infection based on the prediction that an infection is likely. The predetermined threshold may vary based on a recipient or recipient the potential donor is likely to donate stems cells to in the HSCT. The predetermined threshold may be lower if the potential HSCT donor will likely donate stem cells to be used in an HSCT for a younger recipient or recipient who is less sick. In some embodiments, the predetermined threshold is based on a recommendation from a physician, or the user of the methods as described herein.

### D. Potential Applications

In some embodiments, the methods described herein may comprise generating a HSCT blood donation product from the potential HSCT donor if the potential HSCT donor is sorted as a donor. The methods may further comprise treating the potential donor with a mobilization agent to mobilize hematopoietic stem cells from bone marrow to peripheral blood if the potential HSCT donor is a donor. In some embodiments, the mobilization agent is granulocyte-colony stimulating factor (G-CSF).

The HSCT blood donation product may be a post-mobilization HSCT product. The HSCT product may be of higher quality if the potential HSCT donor is identified as a donor using the methods described herein. The quality of an HSCT blood donation product may be determined based on the number of stem cells (e.g. CD34+ cells). In some embodiments, the quality of mobilization is monitored after administration of a mobilization agent to the potential donor. A minimum peripheral blood CD34+ count of 10-20 CD34+ cells per microliter (μL) may considered the minimum threshold to collect a HSCT product.

Accordingly, the methods may comprise treating the potential donor with a mobilization agent to ensure the collection of an adequate number of hematopoietic stem cells (HSCs) from the donor's peripheral blood for hematopoietic stem cell transplantation (HSCT). The mobilization may increase the number of CD34+ stem cells in the bloodstream to facilitate effective collection through apheresis as a HSCT product.

In some embodiments, the HSCT blood donation product generated according to the methods described herein is a high quality HSCT blood donation product. In some embodiments, the high quality HSCT blood donation product contains about 2-5×10⁶ CD34+ cells per kilogram of the recipient's body weight as part of a total about 2-3×10⁸ total cells per kilogram of the recipient's body weight, with at least about 80% viability of the total cells. In some embodiments, the high quality HSCT blood donation product is classified as high quality using additional parameters, such as but not limited the number of mononuclear cells, sterility, and absence of any contaminating tumor cells if the transplant is autologous.

In some embodiments, the methods described herein comprise matching the potential HSCT donor to a recipient in need of a HSCT donation is the potential HSCT donor is sorted as a donor. The methods may further comprise transplanting cells from the potential HSCT donor to the recipient in need of a HSCT donor. The recipient in need of an HSCT donation may be a patient diagnosed with condition likely to improve with an HSCT, such as but not limited to a malignant, non-malignant, or a genetic disorder.

The patient may be diagnosed with a hematologic malignancy, characterized by abnormal proliferation of blood cells, and may require HSCT for curative or remission-inducing purposes. Specific conditions may include leukemia, such as acute, lymphoblastic leukemia (ALL), acute myeloid leukemia (AML), and chronic myeloid leukemia (CML), lymphoma such as Hodgkin's lymphoma and non-Hodgkin's lymphoma, and multiple myeloma. The patient may have a condition requiring high dose chemotherapy, such conditions including neuroblastoma, germ cell tumors and those undergoing myoablative therapy. The patient may be diagnosed with non-malignant disorder that can be treated with HSCT include aplastic anemia, myelodysplastic syndromes (MDS) and Fanconi anemia, thalassemia, sickle cell disease, immune deficiencies such as severe combined immunodeficiency (SCID).

### III. Methods for Training a Machine Learning Model to Predict the Probability of an Outcome

Provided herein are methods that can be used to train a machine learning model to predict the probability of an outcome following a HSCT. The training methods disclosed herein can be used to train the machine learning models described herein for use in sorting a potential HSCT donor as a donor or a non-donor, such as the first machine learning model, the second machine learning model, or the third machine learning model. The methods disclosed herein can be used to train a machine learning model to predict the probability of any of the outcomes described herein following a HSCT. It is contemplated that training a machine learning model for various outcomes related to an HSCT according to the methods described herein can help provide insight into the connections between the immune system and HSCT success. For example, it may be possible to understand the features of an immune profile that relate to rejection, infection, and survival following HSCT. In some aspects, the same features may relate to multiple outcomes and thus a predictive causal chain can be built.

The methods described herein may be used to train a first machine learning model to predict the probability of a positive first outcome following a HSCT. The methods comprising obtaining fluorescent intensity data, generated from a plurality of fluorescently labeled cells from a donor of a plurality of donor individuals who have donated hematopoietic stem cells to a recipient individual of a plurality of recipient individuals; obtaining one or more indications of the positive first outcome of a matched recipient individual following an HSCT from a donor in the plurality of donor individuals; training a machine learning model to predict the probability of a positive first outcome following a HSCT, wherein the training is based at least on (a) a subset of the fluorescent intensity data, or data derived therefrom, for the plurality of donor individuals, and (b) one or more indications of the positive first outcome of a matched recipient following an HSCT from the plurality of donor individuals. In some embodiments, the positive first outcome is survival.

The methods described herein may be used to train a second machine learning model to predict the probability of a second outcome following a HSCT. The methods comprising obtaining fluorescent intensity data, generated from a plurality of fluorescently labeled cells from a donor of a plurality of donor individuals who have donated hematopoietic stem cells to a recipient individual of a plurality of recipient individuals; obtaining one or more indications of the second outcome of a matched recipient individual following an HSCT from a donor in the plurality of donor individuals; training a machine learning model to predict the probability of a second outcome following a HSCT, wherein the training is based at least on (a) a subset of the fluorescent intensity data, or data derived therefrom, for the plurality of donor individuals, and (b) one or more indications of the positive first outcome of a matched recipient following an HSCT from the plurality of donor individuals. In some embodiments, the second outcome is relapse. In some embodiments, the results of the second machine learning model are used to train the first machine learning model.

The methods described herein may be used to train a third machine learning model to predict the probability of a negative first outcome following a HSCT. The methods comprising obtaining fluorescent intensity data, generated from a plurality of fluorescently labeled cells from a donor of a plurality of donor individuals who have donated hematopoietic stem cells to a recipient individual of a plurality of recipient individuals; obtaining one or more indications of the negative first outcome of a matched recipient individual following an HSCT from a donor in the plurality of donor individuals; training a machine learning model to predict the probability of a negative first outcome following a HSCT, wherein the training is based at least on (a) a subset of the fluorescent intensity data, or data derived therefrom, for the plurality of donor individuals, and (b) one or more indications of the negative first outcome of a matched recipient following an HSCT from the plurality of donor individuals. In some embodiments, the negative first outcome is death.

A. Obtaining training data

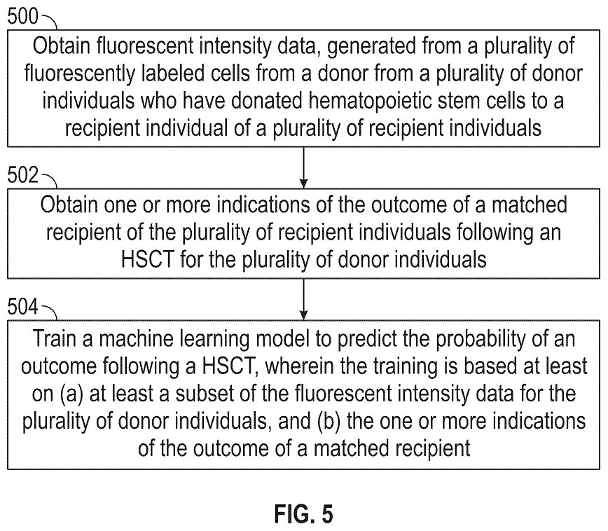

An exemplary embodiment, according to the methods described herein is described in FIG. 5. At block 500, fluorescent intensity data is obtained. The fluorescent data is generated plurality of fluorescently labeled cells from the plurality of donor individuals who have donated hematopoietic stem cells to a recipient individual of a plurality of recipient individuals. The fluorescent intensity data may be generated according to the methods described herein wherein the sample is a sample from a donor in the plurality of donors. In some embodiments, block 500 may comprise fluorescently labeling cells contained within a sample from the donor, by contacting at least an aliquot of the sample with at least one immunophenotyping fluorescent labeling panel and generating fluorescent intensity data by processing the fluorescently-labeled cells from the sample using a flow cytometer according to the methods described herein. In some embodiments, block 500 may comprise generating the fluorescent intensity data according to the methods described herein.

In some embodiments, the sample comprises a pre-mobilized sample. The pre-mobilized sample may have been collected from the donor before the donor received a mobilization agent. In some embodiments, the sample comprises a post-mobilized sample. A post-mobilized sample may be a sample collected from the HSCT donor after the donor was treated with a mobilization agent to mobilization agent to mobilize hematopoietic stem cells from bone marrow to peripheral blood. In some embodiments, the mobilization agent is granulocyte-colony stimulating factor (G-CSF). In some embodiments, the post-mobilized sample comprises stem cells. In some embodiments, the stem cells are CD34+ cells. In some embodiments, the stem cells are hematopoietic stem cells.

In some embodiments, the plurality of donor individuals comprises at least 10, at least 20, at least 30, at least 40, at least 50, at least 60, at least 70, at least 80, at least 90 at least 100, at least 200, at least 300, at least 400, at least 500, at least 600, at least 700, at least 800, at least 900, at least 1000, at least 1500, at least 2000, at least 2100, at least 2200, or at least 2500. In some embodiments, the plurality of donor individuals comprises between about 10 and about 100 individuals, about 20 and 100 individuals, about 40 and 100 individuals, 50 and 100 individuals 60 and 100 individuals, or 100 more individuals. In some embodiments, the plurality of donor individuals comprises between 1000 and 2500, between 1000 and 2000, or between 1000 and 1500 individuals.

At block 502, one or more indications of the outcome of a matched recipient individual following an HSCT from a donor of the plurality of donor individual is obtained. The recipient individual is an individual of the plurality of recipient individuals.

In some embodiments, the one or more indications of the outcome comprise one or more indications of an outcome at multiple timepoints. The multiple timepoints may be any timepoint in a defined time frame. In some embodiments, the defined timeframe may be any of the defined timeframes described herein. In some embodiments, the multiple time points may be daily, monthly, or yearly.

In some embodiments, the individual in the plurality of recipient individuals have each received an HSCT within a defined timeframe, such as any of the defined timeframes described herein. The one or more indications of an outcome may comprise a binary categorization. In some embodiments, the indication of an outcome is a yes if the recipient individual experienced the outcome and a no if the recipient individual did not. In some embodiments, the indication comprises additional information about the outcome such as but not limited to timing, severity, and related clinical information about the recipient individual.

In some embodiments, the individual in the plurality of recipient individuals is a donor in the plurality of donor individuals. A donor can also be a recipient when the HSCT is an autologous transplant. In an autologous transplant healthy cells can be stimulated and donated back to the individual as a treatment.

In some embodiments, the recipient individuals in the plurality of recipient individuals received an HSCT as treatment of a disease. In some embodiments, each individual received the HSCT to treat the same disease. In some embodiments, each individual received the HSCT to treat different diseases. The diseases may be any disease indication wherein an HSCT is used as a treatment. In some embodiments, the disease is a form of cancer such as various hematologic cancers including acute myeloid leukemia (AML), acute lymphoblastic leukemia (ALL), chronic myeloid leukemia (CML), Hodgkin lymphoma, non-Hodgkin lymphoma, and multiple myeloma.

In some embodiments, the plurality of recipient individuals comprises at least 10, at least 20, at least 30, at least 40, at least 50, at least 60, at least 70, at least 80, at least 90 at least 100, at least 200, at least 300, at least 400, at least 500, at least 600, at least 700, at least 800, at least 900, at least 1000, at least 1500, at least 2000, at least 2100, at least 2200, or at least 2500. In some embodiments, the plurality of recipient individuals comprises between about 10 and about 100 individuals, about 20 and 100 individuals, about 40 and 100 individuals, 50 and 100 individuals 60 and 100 individuals, or 100 more individuals. In some embodiments, the recipient of donor individuals comprises between 1000 and 2500, between 1000 and 2000, or between 1000 and 1500 individuals. In some embodiments, the plurality of recipient individuals comprises fewer individuals than the plurality of donor individuals because a single donor individual may have donated to more than one recipient individual.

It is contemplated that the steps recited in blocks 500 and 502 can be performed in parallel or sequentially such that block 500 may be performed before 502 or block 502 may be performed before block 500.

### B. Training the Machine Learning Model

In some embodiments, the machine learning models described herein are trained with one or more indications of the outcome of a matched recipient following an HSCT for the plurality of donor individuals. In some embodiments, the matched recipient is a recipient in a plurality of recipient individuals who has received a HSCT using a blood product derived from a donor in the plurality of donor individuals.

At block 504 a machine learning model is trained to predict probability of an outcome following a HSCT. The training is based at least on a subset of the fluorescent intensity data, or data derived therefrom, for the plurality of donor individuals, and (b) one or more indications of the outcome of a matched recipient following an HSCT from the plurality of donor individuals.

In some embodiments, the machine learning has been trained with two or more indications of the outcome collected at two or more timepoints. In some embodiments, the two or more timepoints may be any timepoints in a predefined timeframe as described herein. In some embodiments the two or more timepoints are daily, monthly, or yearly. In some embodiments, the machine learning model has been trained with two or more indications of the outcome collected at two or more timepoints to output a predicted probability comprising the predicted probability of the outcome at each of the two or more timepoints.

The machine learning model may comprise any of the machine learning model architectures as described herein. The machine learning model may decision tree based classification model or a neural network as described herein. In some embodiments, the machine learning model comprises a decision tree based classification model. In some embodiments, the machine learning model comprises an XGBoost model. In some embodiments, the machine learning model comprises a logistic regression model. In some embodiments, the machine learning model comprises a Adaptive Best Subset Selection Ensemble (ABSSE) model. In some embodiments, training the ABSSE model comprises multilayer feature selection. In some embodiments, training the ABSSE model comprises generating an ensemble of machine learning models. In some embodiments, training the machine learning model comprises selecting two or more feature sets, training two or more machine learning model with the two or more feature sets.

The training at block 504 may comprise any of the training may comprise any of the machine learning model training methods as described herein. In some embodiments, training comprises optimizing performance using hyperparameter tuning as described herein.

In some embodiments, the training may be based on the results of an additional machine learning model trained to predict the probability of a second outcome following a HSCT. In some embodiments, the results of the additional machine learning model comprise features selected by the additional machine learning model as predictive of the outcome the additional machine learning model is trained to predict. In some embodiments, the outcome is a second outcome.

In some embodiments, the methods may comprise training multiple machine learning models with data from different subsets of matched donor and recipients from the plurality of donors and recipients. The subsets may be selected based on characteristics of the donor or recipient individuals. A model may be trained using data from recipient individuals who have been treated with the same disease. A model may be trained using data from donor individuals who share a characteristic, such as a characteristics known in the art to be associated with differences in HSCT success. As described herein, training machine learning models with data from different subsets of individuals may improve the accuracy of a prediction generated with the machine learning model.

### IV. Methods for Training and Using a Mobilization Machine Learning Model to Predict Donor Response to a Mobilization Agent

As described herein mobilization of stem cells from donor bone marrow to their peripheral blood is done before a blood product can be collected and used for HSCT. The quality of an HSCT blood product and the success of the transplant thus relies on effective mobilization. Poor mobilization can lead to delayed engraftment, increased post-transplant complications, or the need for additional mobilization and collection attempts.

At present, there are no reliable methods for determining which donors may successfully mobilize sufficiently for use in HSCT other than basic health and prior treatment factors. Provided herein are methods of training a machine learning model to predict if a donor cells will successfully mobilize. As mobilization is expensive, time consuming and associated with significant side effects in the donors, there is a need for a method for determining which potential donors may successfully mobilize prior to initiation of the mobilization protocol. The described machine learning model can be used to predict success in mobilization using a sample collected before the donor is treated with a mobilization agent. Like the methods described above, the methods allow for screening of blood banks for potential donors that are likely to respond well to mobilization. As described herein, the methods can also be used in parallel with methods for sorting a potential HSCT donor as a donor or a non-donor.

Provided herein are methods of training a machine learning model to predict donor response a mobilization agent. The machine learning model may be the mobilization model as described herein. In some embodiments, the methods comprise: obtaining pre-mobilized fluorescent intensity data, generated from a plurality of fluorescently labeled cells from a pre-mobilized sample collected from a donor of a plurality of donor individuals; obtaining post-mobilized fluorescent intensity data, generated from a plurality of fluorescently labeled cells from a post-mobilized sample collected from the donor; obtaining one or more indications of the response of the donor to a mobilization agent from the post-mobilized fluorescent intensity data; and training a machine learning model to predict donor response to a mobilization agent, wherein the training is based at least on (a) a subset of the pre-mobilized fluorescent intensity data, or data derived therefrom, for the plurality of donor individuals, and (b) one or more indications of the response.

### A. Obtaining Training Data

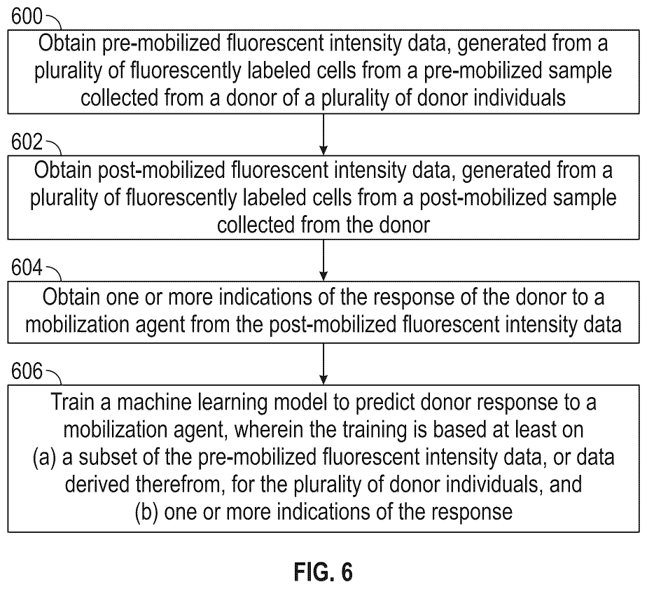

An exemplary embodiments, according to the methods described herein is described in FIG. 6. At block 600, pre-mobilized fluorescent intensity data is obtained. Pre-mobilized fluorescent intensity data is generated according to the methods described herein for generating fluorescent intensity data. Fluorescent intensity data is characterized as pre-mobilized fluorescent intensity data when it is generated from a plurality of fluorescently labeled cells from a pre-mobilized sample. In some embodiments, the pre-mobilized sample is collected from a donor who has donated hematopoietic stem cells to a recipient individual of a plurality of recipient individuals. In some embodiments, outcomes are known for the recipient individual. In some embodiments, the donor has received treatment with a mobilization agent, but a product has not been donated to an individual. In some embodiments, the pre-mobilized sample is a pre-mobilized sample as described herein. The methods may comprise treating an individual with a mobilization agent at block 600 so that a post-mobilized sample and data derived therefrom can be generated or obtained.

At block 602, post-mobilized fluorescent intensity data is obtained. Post-mobilized fluorescent intensity data is generated according to the methods described herein for generating fluorescent intensity data. Fluorescent intensity data is characterized as post-mobilized fluorescent intensity data when it is generated from a plurality of fluorescently labeled cells from a post-mobilized sample. In some embodiments, the post-mobilized sample is collected from a donor after the donor has been treated with a mobilization agent. In some embodiments, the post-mobilized sample has been used for a HSCT to a recipient individual. In some embodiments, outcomes are known for the recipient individual.

In some embodiments, the post-mobilized sample is a post-mobilized sample as described herein. In some embodiments, the post-mobilized sample comprises stem cells. In some embodiments, the post-mobilized sample comprises CD34+ cells.

At block 604, one or more indication of the response of the donor to a mobilization agent is obtained from the post-mobilized fluorescent intensity data obtained at block 602. The one or more indications of the donor to a mobilization agent may comprise the number of CD34+ cells. The one or more indications of the donor to a mobilization agent may comprise the number of stem cells. The one or more indications of the donor to a mobilization agent may comprise the number of cells from any of the cell population related to successful mobilization. The one or more indications of the donor to a mobilization agent may comprise the concentration of CD34+ cells. The concentration may be the number of CD34+ cells per μL of the sample used to generate the fluorescent intensity data. The one or more indications of the donor response to a mobilization agent may comprise the concentration of stem cells. The concentration may be the number of stem cells per μL of the sample used to generate the fluorescent intensity data.

The one or more indications of the donor response to a mobilization agent may comprise a binary indication. In some embodiments, the one or more indication of the donor response may comprise a YES if the number of stem cells is above a threshold, and a NO if the number of stem cells is not above the threshold. The threshold may be determined using teaching regarding the for the number of stem cells needed for a quality HSCT blood product. In some embodiments, the one or more indication of the donor response may comprise a YES if the number of CD34+ is above a threshold, and a NO if the number of CD34+ is not above the threshold. The threshold may be determined using teaching regarding the for the number of CD34+ needed for a quality HSCT blood product.

In some embodiments, the one or more indication of the donor response may comprise a YES if the change in the number of stem cells estimated from the pre-mobilized fluorescent intensity data and the post-mobilized fluorescent intensity data increases and a NO if the number of stem cells does not increase. In some embodiments, the one or more indication of the donor response may comprise a YES if the change in the number of CD34+ estimated from the pre-mobilized fluorescent intensity data and the post-mobilized fluorescent intensity data increases and a NO if the number of CD34+ does not increase.

In some embodiments the one or more indication of the response of the donor to a mobilization agent may comprise a relative change in one or more cell populations between the cell populations identified using the pre-mobilized fluorescent intensity data from block 600 and the post-mobilized fluorescent intensity data from block 602. The one or more cell populations may comprise cell types known in the art to relate to successful mobilization such as but not limited to CD34+ cells.

In some embodiments, the methods may comprise generating one or more indications of the response from the post-mobilized fluorescent intensity data by analyzing the post-mobilized fluorescent intensity data according to the methods described herein. In some embodiments, the methods may comprise generating one or more indications of the response from the post-mobilized fluorescent intensity data by analyzing and comparing the pre-mobilized fluorescent intensity data and post-mobilized fluorescent intensity data according to the methods described herein.

### B. Training the Machine Learning Model

At block 606, a machine learning model is trained to predict donor response to a mobilization agent, wherein the training is based at least on (a) a subset of the pre-mobilized fluorescent intensity data from block 600, or data derived therefrom, for the plurality of donor individuals, and (b) one or more indications of the response from block 604. The predicted response to a mobilization agent may be a probability that the one or more indications of the response as described herein will occur.

The machine learning model may comprise any of the machine learning model architectures as described herein. The machine learning model may decision tree based classification model or a neural network as described herein. In some embodiments, the machine learning model comprises a decision tree based classification model. In some embodiments, the machine learning model comprises an XGBoost model. In some embodiments, the machine learning model comprises a logistic regression model. In some embodiments, the machine learning model comprises a Adaptive Best Subset Selection Ensemble (ABSSE) model.

The training at block 606 may comprise any of the training may comprise any of the machine learning model training methods as described herein. In some embodiments, training comprises optimizing performance using hyperparameter tuning as described herein.

In some embodiments, the training may be based on information about the cellular composition of the post-mobilized sample. In some embodiments, the information about the cellular composition of the post-mobilized sample may be generated from data other than the post-mobilized fluorescent intensity data. In some embodiments, the information about the cellular composition may comprise cell count and or cell viability of one or more cell populations.

In some embodiments, the training may be based on information about the donor individual. The information may be related to demographic information about the individual or health information about the donor individual. In some embodiments, the information used in training may be encoded as covariables when the target variable is the one or more indication of the response.

### C. Using the Machine Learning Model

In some embodiments, the methods described herein comprise predicting a response to a mobilization agent for a potential HSCT donor such as a potential HSCT donor described herein. In some embodiments, the methods are for sorting a potential HSCT donor as a donor or a non-donor, wherein the sorting is based on the output of the mobilization model. In some embodiments, the output of the mobilization model is a predicted response to a mobilization agent. In some embodiments, a potential HSCT donor is sorted as a donor if the predicted response to a mobilization agent is a response associated with production of a high quality HSCT blood product as described herein.

In some embodiments, the model may be used to predict the number of stem cells that will be in a HSCT blood product generated from the potential HSCT donor. In some embodiments, a potential HSCT donor may be sorted as a donor if the predicted number of stem cells is above a threshold. The threshold may be based on the weight of a potential recipient (e.g. 2-5×10⁶ stem cells per kilogram of the recipient's body weight as part of a total 2-3×10⁸ total cells per kilogram of the recipient's body weight). In some embodiments, the model may be used to predict the probability the potential HSCT donor will produce a blood product with sufficient stem cells to generate an HSCT blood product. In some embodiments, the model may be used to predict the probability the potential HSCT donor will produce a high quality HSCT blood product as described herein.

It is appreciated that the methods for sorting a potential HSCT donor as a donor or non-donor based on the results of a first machine learning model, a second machine learning model, or the third machine learning model as described herein may be used to sort an individual based on the results of the mobilization model because success in mobilization is directly related to success in a HSCT. The potential applications described herein for use of the methods for sorting a potential HSCT donor may also be used in combination with methods of predicting donor response to a mobilization agent.

### V. Fluorescent Intensity Data

Provided herein are methods comprising generating and using an immune profile for an individual or plurality of individuals. The immune profile may comprise fluorescent intensity data as described herein. The methods comprise fluorescently labeling cells contained within a sample from a potential HSCT donor or plurality of donor individuals who have donated hematopoietic stem cells to a recipient individual of a plurality of recipient individuals, by contacting at least an aliquot of the sample with at least one immunophenotyping fluorescent labeling panel and generating fluorescent intensity data by processing the fluorescently-labeled cells from the sample using a flow cytometer. In some embodiments, the methods comprise obtaining fluorescent intensity data, generated from a plurality of fluorescently labeled cells from the potential HSCT donor plurality of donor individuals who have donated hematopoietic stem cells to a recipient individual of a plurality of recipient individuals.

In some embodiments, the fluorescent intensity data is obtained using flow cytometry. In some embodiments, the fluorescent intensity data are processed into flow cell classifications. In some embodiments, the fluorescent intensity data is obtained using flow cytometry followed by machine learning models to analyze cells and classify them using a standardized set of immune system status antibody panels. In some embodiments, the flow cytometer outputs cells classifications. In some embodiments, the flow cytometer outputs mean fluorescent intensity (MFI) data.

In some embodiments, the fluorescent intensity data is obtained using flow cytometry. In some embodiments, the fluorescent intensity data is generated using a flow cytometry to process fluorescently labeled cells from the sample. In some embodiments, the flow cytometer is configured for at least about 5, at least about 10, at least about 15, at least about 20, at least about 30, at least about 40, at least about 50, at least about 60, at least about 70, at least about 80, at least about 90, or at least about 100 fluorescent detection channels. In some embodiments, the flow cytometer is configured for between about 5 and about 100, between about 10 and about 90, between about 20 and about 80, between about 30 and about 70, or between about 40 and about 60 fluorescent detection channels. In some embodiments, the flow cytometry is a full spectrum flow cytometer.

In some embodiments, flow cytometry is performed on sample. In some embodiments, the samples are received at the laboratory facility, the sample is prepared and analyzed with flow cytometry. In some embodiments, preparing the sample comprises performing one or more of a dilution step, a centrifugation step, a staining step (using one or more fluorescently-labeled antibody panels) and/or a wash step.

In some embodiments, the staining step comprises contacting cells contained within a sample from a potential HSCT donor or a donor from a plurality of HSCT donors with at least one immunophenotyping fluorescent labeling panel (i.e., an immunophenotyping panel or flow cytometry panel). In some embodiments, the one immunophenotyping fluorescent labeling panel comprises fluorescently-labeled antibodies directed to a set of specific cell surface antigens (e.g., cell surface proteins) that collectively enable discrimination between the cell types or cell subtypes of interest. Sample processing may also include immunophenotyping panel design. A sample processing platform may comprise contacting each of one or more sample aliquots (e.g., 1, 2, 3, 4, 5, 6, 7, 8, 9, 10, or more than 10 sample aliquots) with one or more flow cytometry panels (e.g., 1, 2, 3, 4, 5, 6, 7, 8, 9, 10, or more than 10 flow cytometry panels).

In some embodiments, a flow cytometry panel or immunophenotyping panel may comprise at least about 5, at least about 10, at least about 15, at least about 20, at least about 30, at least about 40, at least about 50, at least about 60, at least about 70, at least about 80, at least about 90 or at least about 100 fluorescently-labeled antibodies directed to a set of cell surface antigens. In some embodiments, a flow cytometry panel or immunophenotyping panel may comprise between about 5 and about 100, between about 10 and about 90, between about 20 and about 80, between about 30 and about 70, or between about 40 and about 60 fluorescently-labeled antibodies directed to a set of cell surface antigens.

In some embodiments, cells from each of the sample may be divided into aliquots and the aliquots may be stained with a different flow cytometry panel, one focusing on the antigen-presenting cell (APC) arm of the immune system (A panel), which comprises antibodies directed to a plurality different cell surface proteins, e.g. 36, and the other focusing on the adaptive arm of the immune systems (T panel), which comprises antibodies directed to a second plurality of cell surface markers, e.g. 41 cell surface proteins. In some instances, the panels may also include cell viability staining to distinguish between live cells and dead cells. In some instances, the panels may also comprise an autofluorescence measurement as a “marker”. Non-limiting examples of the cell surface proteins and additional markers that may be included in these panels are listed in Table 1.

TABLE 1
Non-limiting examples of cell surface receptor proteins and other
markers for distinguishing between immune cell sub-populations.

| APC panel markers (A panel) | T panel markers (T panel) |
|---|---|
| IGM, LIVE_DEAD_BLUE, CD5, CD62L, | TIGIT, CD5, CD28, CXCR5, CD39, TIM3, |
| CD294, CD69, CD38, PD1, CD11C, CD3, | CD38, PD1, TCRVA7_2_TCRVD1, CD95, |
| CD8, HLADR, CD24, CD337, CD123, | CD3, CD8, HLADR, CD31, CCR4, CCR6, |
| CD141, Autofluorescence 1, CD1C, CD4, | CCR7, Autofluorescence 1, CD57, ICOS, |
| TACI, CD319, CD335, PDL1, CD10, CD45, | CD4, KLRG1, TCRVA24_JA18, CD122, |
| CD16, IGD, CD40, CD19_TCRGD, CD43, | CD103, CXCR3, TCRVD2, CD45, CCR10, |
| CD14, CD138, CD15, CD56, CD86, CD303, | CD16, CD25, CD161, CD19_TCRGD, |
| CD27 | LAG3, CD14, CD45RO, CD56, CD127, |
| | CD45RA, CD27 |

In some embodiments, the panels include markers for determining immune cell type, immune system activation, lineage (e.g., the main marker(s) that are commonly used to define a certain cell population prior to further subsetting the cell type; examples include, but are not limited to, CD3 to define total T cells, and CD56 and CD16 to define natural killer cells), and exhaustion (cells that express markers associated with “cell exhaustion” (e.g., PD-1, TIGIT) can no longer proliferate and lose their functionalities as a result of chronic stimulation/prolonged activation of immune response).

In some embodiments, the panels include markers such as lineage markers for αβT cells, invariant T cells, γδT cells, B cells, NK cells, monocytes, macrophages, dendritic cells, neutrophils, eosinophils, and basophils. In some embodiments, the lineage markers include CD3, CD4, CD8, CD25, CD45, CD19, CD27, IgD, IgM, CD56, CD16, CD14, HLA-DR, CD11c, CD56, TCRgd, TCR Vα7.2. TCR Vδ1, TCR Vδ2, TCR Vα24-Jα18.

In some embodiments, the panels include markers such as functional markers relating to but not limited to activation, migration, exhaustion, senescence, or memory status of cells. In some embodiments, the functional markers include CCR10, CD103/ITGAE, CD122/IL2RB, CD161/KLRB1, CD223/LAG-3, CD274/PD-L1, CD335/NKp46, CD43, CD10, CD138, CD141, CD183/CXCR3, CD185/CXCR5, CD194/CCR4, CD197/CCR7, CD279/PD-1, CD28, CD294/CRTH2, CD337/NKp30, CD38, CD39, CD5, CD62L, CD86, CD95, ICOS, TIGIT, TIM-3, CD40, KLRG1, CD69, CD196/CCR6, CD1c, CD24, CD267/TACI, CD303/BDCA-2/CLEC4C, CD31, CD319, CD57, CD127, CD45RO, CD45RA.

In some embodiments, the fluorescent intensity data is obtained using flow cytometry and is in the form of a flow cytometry standard FCS file. In some embodiments the flow cytometry data comprises mean fluorescent intensity (MFI) data. In some embodiments, the FCS file may comprise, for example, fluorescence intensity data for one or more fluorescence detection channels (e.g., 2, 3, 4, 5, 6, 7, 8, 9, 10, 11, 12, 13, 14, 15, 20, 25, 30, 35, 40, 50, or more than 50 fluorescence detection channels, as well as data derived therefrom (e.g., forward scatter height data, forward scatter area data, side scatter height data, side scatter area data, autofluorescence data, or any combination thereof). In some instances, the number of fluorescence detection channels available may be determined by, for example, a combination of the detection hardware available as part of the flow cytometry instrument (e.g., comprising 5, 10, 20, 25, 50, 75, 100, 125, 150, 175, 200, or more than 200 detectors) and the number of spectrally-distinct fluorophores (e.g., 5, 10, 20, 25, 30, 35, 40, 45, 50, 60, or more than 60 spectrally-distinct fluorophores). In some embodiments, manual gating may be used to determine flow cell classification for the plurality of cells. In some embodiments, manual gating results in about 10-2500, about 200-2500, about 1000-2500 cell classification, or 2000-2300 cell classification. In some embodiments, manual gating may be performed by an expert, e.g. an immunologist. In some embodiments, the cell classification may relate to cell types, cell subtypes, or cell states.

In some embodiments, trained machine learning models may be used to determine flow cell classifications for the second plurality of cells. In some embodiments, the models trained using the method described in U.S. Ser. No. 18/353,022. In some embodiments, trained machine learning models are used to produce predictions of cell type or subtype (e.g., immune cell sub-population) for individual cell detection events and to determine cell counts (or frequencies) for each of a plurality of distinct cell types or subtypes. In some embodiments, the trained machine learning model uses a common hierarchy (a.k.a, a gating tree) to process the fluorescence profile data for each detected event and determine which and how many events belong to each measured populations (e.g., immune cell sub-population) in the hierarchy. In some embodiments, this may comprise over about 200 gates for the APC panel and over about 2000 gates for the T cell panel. The advantages of using this approach can be found in U.S. Ser. No. 18/353,022 incorporated by reference in its entirety.

In some embodiments, a gating tree is constructed. In some embodiments, the gating tree comprises cell subsets. In some embodiments, the cell subsets are CD45+ (Leukocytes), CD3+ (T cells), CD4+ (Helper T cells), CD8+ (Cytotoxic T cells), CD19+ (B cells), CD14+ (Monocytes), CD56+ (Natural Killer cells), CD16+ (Neutrophils), HLA-DR+ (Activated T cells), CD3-CD56+ (NK cells), CD3-CD19+ (Non-T/Non-NK B cells), CD3+CD16+ (NKT cells), CD4+CD45RA+ (Naïve T cells), CD4+CD45RO+ (Memory T cells), CD8+CD45RA+ (Naïve Cytotoxic T cells), CD8+CD45RO+ (Memory Cytotoxic T cells), CD14+HLA-DR+ (Activated Monocytes), CD16+CD45+ (Granulocytes), CD3-CD19-HLA-DR+ (Non-T/Non-B/Non-NK activated cells), CD3+HLA-DR+ (Activated T cells).

In some embodiments, the fluorescent intensity data, or data derived therefrom, comprises cell classifications. In some embodiments, the cell classification data comprises summary ratio data for the cell classifications identified using the methods described herein.

In some embodiments, the fluorescent intensity data may comprise immune cell populations. In some embodiments, the immune cell populations are distinct immune cell populations. In some embodiments, the immune cell populations are 20 distinct immune cell populations.

### VI. HSCT Outcomes

The methods provided herein comprise machine learning models trained to predict the probability of an outcome following an HSCT, such as a first outcome, a positive first outcome, a negative first outcome, or a second outcome. The machine learning models can be used to generate a predicted probability of an outcome following an HSCT, such as a first outcome, a positive first outcome, a negative first outcome, or a second outcome in order to sort a potential HSCT donor as a donor (e.g. universal donor) or a non-donor. It is contemplated that the methods described herein may pertain to a variety of outcomes that may be used to indicate the success for an HSCT in a recipient individual. The outcomes may relate to complications with an HSCT, relapse of a disease, or death of the recipient.

In some embodiments, the outcome is survival. In some embodiments, the outcome is survival between the HSCT and a defined timeframe. In some embodiments, the positive first outcome is survival between the HSCT and a defined timeframe. In some embodiments, the negative first outcome is death between the HSCT and a defined timeframe when the positive first outcome is survival between the HSCT and a defined timeframe. In some embodiments, the second outcome is survival between the HSCT and a defined timeframe. In some embodiments, the second outcome is death between the HSCT and a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

In some embodiments, the outcome is lack of infection. In some embodiments, the outcome is lack of infection between the HSCT and a defined timeframe. In some embodiments, the positive first outcome is lack of infection between the HSCT and a defined timeframe. In some embodiments, the negative first outcome is infection between the HSCT and a defined timeframe when the positive first outcome is lack of infection between the HSCT and a defined timeframe. In some embodiments, the second outcome is lack of infection between the HSCT and a defined timeframe. In some embodiments, the second outcome is infection between the HSCT and a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

In some embodiments, the outcome is lack of graft-versus-host disease (GvHD). In some embodiments the GvHD is acute GvHD. In some embodiments, the GvHD is chronic GvHD. Acute GvHD occurs in 30% to 50% of matched sibling transplants and up to 70% in unrelated donor transplants. Chronic GvHD affects 30% to 70% of recipients and can impair graft function, leading to graft failure in severe cases. Accordingly predicting lack of GvHD is of concern for choosing a potential donor for HSCT.

In some embodiments, the outcome is lack of GvHD between the HSCT and a defined timeframe. In some embodiments, the positive first outcome is lack of GvHD between the HSCT and a defined timeframe. In some embodiments, the negative first outcome is GvHD between the HSCT and a defined timeframe when the positive first outcome is lack of GvHD between the HSCT and a defined timeframe. In some embodiments, the second outcome is lack of GvHD between the HSCT and a defined timeframe. In some embodiments, the second outcome is GvHD between the HSCT and a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

In some embodiments, the outcome is lack of graft rejection. In some embodiments, graft rejection may be due to host-versus-graft reactions or pre-existing alloantibodies. As failure rates range from 5% to 20% depending on HLA matching and conditioning regimens, predicting graft rejection or the absence of graft rejection is of concern in choosing a potential donor for HSCT.

In some embodiments, the outcome is lack of graft rejection between the HSCT and a defined timeframe. In some embodiments, the positive first outcome is lack of graft rejection between the HSCT and a defined timeframe. In some embodiments, the negative first outcome is graft rejection between the HSCT and a defined timeframe when the positive first outcome is lack of graft rejection between the HSCT and a defined timeframe. In some embodiments, the second outcome is lack of graft rejection between the HSCT and a defined timeframe. In some embodiments, the second outcome is graft rejection between the HSCT and a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

In some embodiments, the outcome is Immune Reconstitution Failure (IRF). Immune Reconstitution Failure may comprise a delayed immune recovery which affects up to 20% of recipients. Delated immune recovery may compromise engraftment and increase infection risks making it an important outcome to understand and predict in potential HSCT donors.

In some embodiments, the outcome is lack of IRF between the HSCT and a defined timeframe. In some embodiments, the positive first outcome is lack of IRF between the HSCT and a defined timeframe. In some embodiments, the negative first outcome is IRF between the HSCT and a defined timeframe when the positive first outcome is lack of IRF between the HSCT and a defined timeframe. In some embodiments, the second outcome is lack of IRF between the HSCT and a defined timeframe. In some embodiments, the second outcome is IRF between the HSCT and a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

In some embodiments, the outcome is lack of disease relapse. As described above HSCT is used to treat various forms of cancer such as various hematologic cancers including acute myeloid leukemia (AML), acute lymphoblastic leukemia (ALL), chronic myeloid leukemia (CML), Hodgkin lymphoma, non-Hodgkin lymphoma, and multiple myeloma. The procedure involves infusing healthy donor hematopoietic stem cells into the patient to restore the bone marrow's ability to produce blood cells after the bone marrow has been destroyed by high-dose chemotherapy and/or radiation therapy. Relapse rate following HSCT can vary depending on the type and stage of disease. Furthermore, allogeneic transplants can induce a graft-versus-tumor effect where the donor immune cells attack remaining cancer cells, potentially reducing relapse rates. For example, in acute myeloid leukemia (AML), the relapse rate after autologous HSCT can be as high as 50%, while allogeneic HSCT may have relapse rates around 20-30%, demonstrating the importance of the anti-tumor effect that the donor HSC can provide. Accordingly, predicting treatment success in the form of relapse is of interest for choosing a potential donor for an HSCT.

In some embodiments, the outcome is lack of disease relapse between the HSCT and a defined timeframe. In some embodiments, the positive first outcome is lack of disease relapse between the HSCT and a defined timeframe. In some embodiments, the negative first outcome is disease relapse between the HSCT and a defined timeframe when the positive first outcome is lack of disease relapse between the HSCT and a defined timeframe. In some embodiments, the second outcome is lack of disease relapse between the HSCT and a defined timeframe. In some embodiments, the second outcome is disease relapse between the HSCT and a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

In some embodiments, the outcome is related to positive progress in a graft-versus-tumor effect. Positive progress could be a decrease in the residual disease in the recipient. The positive progress may be measured according to whether the recipient has met a minimal residual disease threshold set by clinical guidelines. Although a recipient may not have met the minimal residual disease threshold for remission, a decrease in residual disease may still be considered positive progress. In some embodiments, the positive outcome is positive progress in a graft-versus-tumor effect. In some embodiments, the negative outcome is no change in the progress or negative progress in a graft-versus-tumor effect. In some embodiments, the positive progress is measured within a defined timeframe. In some embodiments, the defined timeframe is one year. In some embodiments, the defined timeframe is 6 months, 1 year, 2 years, 3 years, 4 years, or 5 years. In some embodiments, the defined timeframe is more than 5 years.

### VII. Machine Learning Model Architectures and Training Methods

Provided herein are methods of training and using machine learning models to predict the probability of an outcome following a HSCT. The outcomes may be any outcome related with the success of an HSCT such as any of the outcomes described herein. As described herein, multiple machine learning models can be trained using one or more outcomes and can be combined to improve performance of a model or inform the sorting of a potential donor as a donor or a non-donor. In some embodiments, the results of a second machine learning model can be used as in training a first machine learning model. In some embodiments, the output of one or more machine learning models can be used in sorting a potential HSCT donor as a donor or a non-donor.

Also provided herein are methods of training and using machine learning models to predict the donor response to a mobilization agent. The donor response to a mobilization agent may be the predicted quality of a post-mobilized sample as described herein. The donor response to a mobilization agent may be a quantification of stem cells in a post-mobilized sample as described herein. The quantification of stem cells may be a quantification of CD34+ cells. As described herein, the machine learning models may be trained with at least a subset of pre-mobilized fluorescent intensity data, or data described therefrom, for a plurality of donor individuals, and one or more indications of the response to mobilization.

In some embodiments, the machine learning models (e.g. first machine learning model, second machine learning model, third machine learning model, or mobilization model) may comprise the same architecture. In some embodiments, the machine learning models (e.g. first machine learning model, second machine learning model, third machine learning model, or mobilization model) may comprise different architectures.

In some embodiments, the machine learning model (e.g. first machine learning model, second machine learning model, third machine learning model, or mobilization model) comprises a decision tree based classification model. In some embodiments, the machine learning model comprises a boosted model, such as a XGBoost model. The input features may be fluorescent intensity data, or data derived therefrom as described herein. In some embodiments, the fluorescent intensity data, or data derived therefrom comprise cell classification. In some embodiments, the fluorescent intensity data, or data derived therefrom comprise ratios of cell classifications.

In some embodiments, the machine learning model (e.g. first machine learning model, second machine learning model, third machine learning model, or mobilization model) comprises a decision tree based classification model trained using labeled data to differentiate donor immune profiles (e.g., fluorescent intensity data, or data derived therefrom) according to the outcome. The input features may be the result of a machine learning model as described herein and/or fluorescent intensity data, or data derived therefrom as described herein. In some embodiments, the fluorescent intensity data, or data derived therefrom comprise cell classification. In some embodiments, the fluorescent intensity data, or data derived therefrom comprise ratios of cell classifications. The target variable may be the outcome, such as a positive first outcome, negative first outcome, and/or a second outcome. In some embodiments, the target variable may be an indication of a response to mobilization. In some embodiments, the target variable is coded as binary.

During training, the model may assemble and compare a plurality of trees. In some embodiments, training the machine learning model comprises generating a plurality of decision trees.

The training may comprise estimating the best possible tree by estimating the best possible reduction in error at each training step. In some embodiments, the error is computed for a first tree and feed forward the next tree in the plurality of trees. In some embodiments, the model's performance is monitored during training with a LogLoss metric. As the trees are ensembled, the LogLoss may decrease as the data is fit. The model's performance during training may be monitored using any metric known in the art, such as but not limited to an estimate of positive correctness, Gini impurity, information gain, variance reduction, or measures of “goodness.”

In some embodiments, training the machine learning model (e.g. first machine learning model, second machine learning model, third machine learning model, or mobilization model) comprises optimizing the performance of the model. In some embodiments, optimizing the performance of the model comprises hyperparameter tuning. Hyperparameter tuning adjusts the model's key parameters to achieve the best predictive accuracy and generalizability to unseen data. In some embodiments, training the machine learning model comprises hyperparameter tuning for the aspects of the XGBoost model, such as the number of decision trees, the learning rate, and the depth of the trees. In some embodiments, the hyperparameter tuning comprises systematically testing multiple combinations of hyperparameters to identify the configuration that results in the best performance. In some embodiments, the hyperparameter tuning comprises Grid Search.

In some embodiments, the machine learning model (e.g. first machine learning model, second machine learning model, third machine learning model, or mobilization model) comprises a RandomForest model. In some embodiments, the RandomForest model selects features that are predictive of the target outcome. In some embodiments, the machine learning model comprises a Support Vector Machine.

In some embodiments, the machine learning model (e.g. first machine learning model, second machine learning model, third machine learning model, or mobilization model) comprises a logistic regression model. The logistic regression model may be configured by scaling features using a MinMaxScaler. In some embodiments, the MinMaxScaler transforms features to a range between 0 and 1. Other methods known in the art may be applied to scale the features. In some embodiments, the logistic regression model is configured by calling features using StandardScaler. In some embodiments, the StandardScaler transforms features to have a mean of 0 and a standard deviation of 1.

In some embodiments, the machine learning model (e.g. first machine learning model, second machine learning model, third machine learning model, or mobilization model) may be organized in a cascading hierarchical tree structure comprising a plurality of nodes. In some embodiments, each node may be an individual machine learning model. In some embodiments, each individual machine learning model may comprise a neural network. In some embodiments, each individual machine learning model may comprise a multi-input neural network. In some embodiments, each individual machine learning model may comprise a deep feature fusion model. In some embodiments, each individual machine learning model may comprise a recurrent neural network (RNN). In some embodiments, each individual machine learning model may comprise a gradient boosting tree model.

In some embodiments, each layer of the neural network comprises a number of nodes (or perceptrons). In some embodiments, a node receives input that comes either directly from the input data (e.g., fluorescent intensity data) or the output of nodes in previous layers, and performs a specific operation, e.g., a summation operation. In some cases, a connection from an input to a node is associated with a weight (or weighting factor). In some cases, the node may, for example, sum up the products of all pairs of inputs from a previous layer and their associated weights. In some cases, the weighted sum is offset with a bias, b. In some cases, the output of a node may be gated using a threshold or activation function, f, which may be a linear or non-linear function. The activation function may be, for example, a rectified linear unit (ReLU) activation function or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some embodiments, the machine learning model (e.g. first machine learning model, second machine learning model, third machine learning model, or mobilization model) may be a neural network trained using labeled data to differentiate donor immune profiles likely to result in an outcome such as any of the outcomes described herein. The labeled data may be fluorescent intensity data, or data derived therefrom as described herein. In some embodiments, the fluorescent intensity data, or data derived therefrom comprise MFI data. In some embodiments, the neural network comprises an input layer, one or more hidden layers, and an output later. In some embodiments, the one or more hidden layers comprise three hidden layers. In some embodiments, the first hidden layer comprises a dense layer with 64 neurons and uses a ReLu activation function to process input data. In some embodiments, the second hidden layer comprises a dense layer with 32 neurons and uses a ReLu activation function to consolidate latent information that may be a signal of a response variable. In some embodiments, the third hidden layer comprises a dense layer with 32 neurons and uses a ReLu activation function to consolidate latent information that may be a signal of a response variable. In some embodiments, the output layer comprises a dense layer with 2 output neurons using a Softmax activation function. In some embodiments, the output layer outputs the distribution over two classes, outcome, non-outcome. In some embodiments, the output layer outputs the probability of input data correlating with the outcome.

In some embodiments, the machine learning model (e.g. first machine learning model, second machine learning model, third machine learning model, or mobilization model) may be a fully connected neural network. In some embodiments, the neural network has 10 inputs, 20 inputs 30 inputs, 40 inputs, 50 inputs, 60 inputs or 70 or more inputs. In some embodiments, the neural network comprises one or more hidden layers. In some embodiments, the one or more hidden layers comprise one or more neurons. In some embodiments, the one or more hidden layers comprise 5 neurons, 10 neurons, 20 neurons, 30 neurons, 40 neurons or 50 or more neurons. In some embodiments, fully connected neural network is a fully connected neural network with about 20 input parameters, 1 hidden layer with 10 or more neurons and an output layer.

In some embodiments, the machine learning model (e.g. first machine learning model, second machine learning model, third machine learning model, or mobilization model) may be a multi-input neural network trained using labeled data to differentiate between outcomes. The labeled data for the models may be fluorescent intensity data, or data derived therefrom as described herein. In some embodiments, the fluorescent intensity data, or data derived therefrom comprise MFI data. In some embodiments, additional measurements such as transcriptomic or genomic data may be used to train the machine learning model. In some embodiments, the one or more clinical measures and one or more demographic measures may be used to increase the sensitivity of the machine learning model. In some embodiments, the one or more clinical measures and one or more demographic measures may be incorporated before or after any of the one or more hidden layers.

In some embodiments, the machine learning model (e.g. first machine learning model, second machine learning model, third machine learning model, or mobilization model) may be a deep feature fusion model trained using labeled data to differentiate between outcomes as well as one or more clinical measures and one or more demographic measures as described herein. In some embodiments, additional measurements such as transcriptomic or genomic data may be used to train the machine learning model. In some embodiments, the one or more clinical measures and one or more demographic measures may be used to increase the sensitivity of the machine learning model.

In some embodiments, the machine learning model (e.g. first machine learning model, second machine learning model, third machine learning model, or mobilization model) may be a machine learning model capable of processing time series data. In some embodiments, time series data may comprise an outcome, such as a positive outcome or a negative outcome collected at two or more timepoints as described herein. The input features for the models may be fluorescent intensity data, or data derived therefrom as described herein. In some embodiments, the fluorescent intensity data, or data derived therefrom comprise MFI data. In some embodiments, the machine learning model may be a recurrent neural network (RNN). In some embodiments, the RNN comprises one or more hidden nodes. In some embodiments, the one or more hidden nodes can be configured as a gated recurrent unit (GRU), a long short-term memory (LSTM) or a combination thereof. In some embodiments, the RNN takes advantage of input data collected at two or more timepoints to enhance decision support for medical interventions. In some embodiments, the RNN can discern patterns indicative of the onset of an outcome such as an infection, disease relapse or HvGD. In some embodiments, the training of the RNN entails systematic adjustment of the network internal weights and biases to minimize prediction errors on training data, ensuring that the model accurately forecasts clinical metrics. It is appreciated that other models known in the art for processing time series data can be used. Such models may include but are not limited to AMIRA, exponential smoothing, and Temporal convolutional neural networks.

In some embodiments, the weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) that the neural network predicts are consistent with the examples included in the training data set. The adjustable parameters of the model may be obtained using, e.g., a back propagation neural network training process.

In some embodiments, the plurality of nodes (i.e., the number of individual machine learning models in the ensemble) comprises at least 1000, 1200, 1400, 1600, 1800, 2000, 2200, 2400, 2600, 2800, 3000, 3200, or 3400 nodes.

In some embodiments, the machine learning model may suffer from overfitting. In some embodiments, hard-parameter sharing may be used to help with mitigation of loss inherent to overfitting models. In some embodiments, hard-parameter sharing may be implementing a deep neural network in which the deeper hidden layers are shared between all tasks (which learn and simultaneously reduce the dimensionality of the information contained in the features), while each target (or biologically-informed group of targets) has dedicated output layers in the network architecture which serve to pseudo-independently predict the expected clinical metrics from the encoded information of the hidden layers.

In some embodiments, a convolutional approach may be employed to capture the inter dependencies. Convolutional neural networks have been shown to be a powerful approach in image analysis, in which pixel-to-pixel correlations are inherent and form shapes that constitute the meaning of the image. CNNs have also been adapted to non-image data, in which the order of the “pixels” (or features and samples in ML nomenclature) do not contain useful information. These approaches require the network to be made agnostic to the order of the input data.

In some embodiments, a multilevel Mixture of Experts approach may be employed where a subset of previously trained models can be used to vote, and a second later discriminatory machine learning model makes the final prediction.

In some embodiments, the machine learning model is compiled with an optimized with an optimizer. In some embodiments, the optimized by adjusting the network's weights to minimize the categorical cross-entropy loss function. In some embodiments, the optimizer is Adam.

In some embodiments, the data used to train the machine learning model may be normalized before it is used to train the machine learning model. In some embodiments, normalizing the data comprises ensuring the data have similar scales. In some embodiments, normalizing the data comprises one-hot-encoding any categorical variables.

In some embodiments, training the machine learning model comprises feeding the data used to train the model into the network, allowing it to adjust its weights over multiple epochs to minimize the loss function. In some embodiments, each epoch represents a full pass through the training dataset. In some embodiments, a validation set is used concurrently to monitor the model's performance and adjust hyperparameters, such as the learning rate and the number of epochs. In some embodiments, early stopping is implemented to prevent overfitting; training is halted if the validation loss does not improve over several epochs.

In some embodiments, the machine learning model (e.g. first machine learning model, second machine learning model, third machine learning model, or mobilization model) described herein comprises an ensemble model. In some embodiments, the ensemble model is used for assessing relapse risk following hematopoietic stem cell transplantation using donor derived materials. In some embodiments, the ensemble model is an Adaptive Best Subset Selection Ensemble (ABSSE) model trained according to the feature subset workflow described herein. The embodiments described below described applying an ABSSE model for predicting disease relapse using donor derived immune profiles. As described herein, disease relapse is a negative outcome. One of skill in the art would understand that the methods can be applied to predict the probability of a positive outcome such as but not limited to survival, lack of infection, lack of disease relapse, or lack of graft vs host disease (GvHD).

In some embodiments, the ABSSE models described herein can be beneficial for predicting probabilities of relapse because of the high dimensionality of the donor cell classification data used for training and inference. In some embodiments in which the ensemble is trained from randomly generated feature subsets, the workflow employs a single stratified training and validation split together with balanced accuracy scoring to avoid data leakage while preserving visibility into feature usage. In further embodiments, the ABSSE models optionally incorporate additional safeguards such as consistency checks during any interaction term creation step and the use of reserved samples to provide unbiased evaluation when those capabilities are desired.

In some embodiments, ABSSE is an iterative ensemble classifier specifically designed for identifying signatures within flow cytometry data and related donor phenotypes, particularly when complex interactions and correlations do not follow the assumptions of conventional classification methodologies. In some embodiments, the methodology integrated into a comprehensive pipeline addresses limitations of traditional feature selection approaches through intelligent sampling strategies and rigorous validation. Optional embodiments further layer adaptive weighting, multistage refinement, or other adaptive learning mechanisms optimized for clinical transplant applications. In some embodiments, ABSSE can be configured to ingest additional clinical or demographic measurements, for example donor age, recipient sex, or conditioning regime.

In some embodiments, an ABSSE workflow comprises selecting cytometry derived features from donor materials, forming candidate feature subsets by random or weighted sampling, fitting a base classifier such as a linear support vector classifier to each subset using a primary train and test split other stratified by relapse outcome, scoring each subset via balanced accuracy computed from cross validation on the training portion together with thresholded predictions on the held out portion and assembling the highest scoring subsets into an ensemble for downstream inference. In some embodiments, the decision threshold may be selected according to practical or clinical considerations, for example to bias against false negative classifications, such as predicting a donor will not lead to relapse when relapse would in fact occur, or to approximate disease prevalence for improved real-world applicability. In certain configurations, a higher threshold reduces false positives but may exclude a greater proportion of true positives (increasing false negatives), whereas a lower threshold increases sensitivity and reduces false negatives, but correspondingly raises the likelihood of false positive classifications. In certain embodiments, the selected feature subsets constitute ensemble blocks that can be stored for later evaluation or deployment.

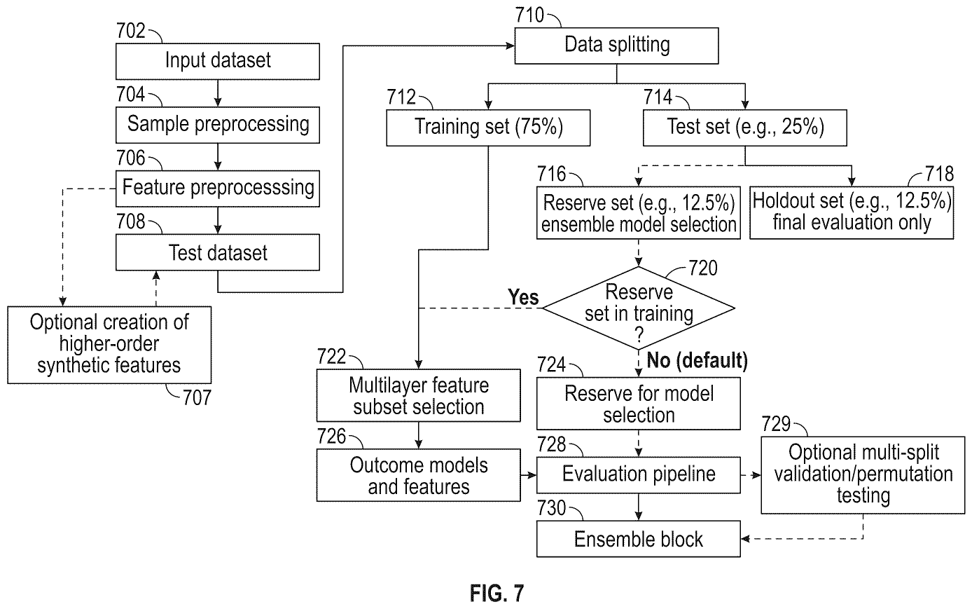

In some embodiments, training an ABSSE model comprises data preparation, multilayer feature selection, ensemble model election, and validation based on unseen data. FIG. 7 provides an exemplary process for training and an ABSSE model as described herein. Input dataset, 702, comprises cell classification data as described herein for patients samples. In some embodiments, samples from the input dataset, 702, are processed at 704.

In some embodiments, additional processing steps can include donor sample quality filtering 704, statistical feature filtering 706, and optional creation of higher-order synthetic features through interaction terms 707 before partitioning 710 into training 712, optional reserve 716, and holdout 718 subsets. In some embodiments, the reserve subset 716 can either be incorporated into feature selection 722 or preserved for model selection 724. Optional embodiments further employ multilayer feature selection 722 that iteratively reweights feature sampling probabilities using reward and penalty updates governed by thresholds. In some embodiments, at each iteration, the selected features are evaluated by training a lightweight classifier, such as a support vector classifier (SVC), on an internal training subset and testing performance on an internal validation subset. The SVC can be replaced with a different machine learning model, such as but not limited to a neural network. A composite balanced accuracy score is calculated using both internal training and test outcomes. The score is compared against the bad threshold and white threshold. If the score exceeds the white threshold, the associated feature weights are increased by the reward value, thereby increasing the likelihood of reselection in subsequent iterations. Conversely, scores below the bad threshold result in a penalty being applied to the weights, reducing the likelihood of reselection. In other embodiments, down sampling, seed resets, batch optimization, or permutation based evaluations are invoked to mitigate overfitting or quantify risk.

In some embodiments, the output of feature selection 722 comprises ranked lists of feature subsets together with their trained models 726. Rankings may rely on geometric mean scores derived from the primary split. In certain embodiments, alternative ranking strategies such as top N, drop point, or weighted drop point analyses are applied to emphasize frequently recurring features among high performing subsets. The “top N” method selects the N top models based on their scores (balanced accuracy) and then identifies the N most frequent features. The “drop point” method first determines the top models exhibiting maximum variation and subsequently selects the N most frequent features. The “weighted drop point”, default, method is similar to the drop point method, but features are weighted by their importance scores.

In some embodiments, evaluation of ensemble candidates relies on the held out portion 718 of the primary split 714. In other embodiments, evaluation may additionally leverage reserve 724 or holdout subsets 718, repeated resampling, or permutation testing 729 to compute risk scores prior to exporting ensemble blocks 730.

In some embodiments, inference using the ABSSE model comprises aggregating predictions from the trained subset models. A default embodiment that averages predicted class probabilities and applies a configurable decision threshold to yield a relapse risk prediction may be used. In other embodiments, majority voting, weighted voting, or logit margin aggregation strategies are employed.

In some embodiments, the stability of the ABSSE ensemble is assessed by retraining the model across multiple resampled or randomly partitioned subsets of the training data and evaluating performance on corresponding held-out sets. In certain implementations, the training dataset is divided into S partitions, and an independent ABSSE model is trained on each partition. The resulting models are then applied to the reserved and holdout subsets, and stability is determined from the balanced accuracy obtained across these replicates.

In alternative embodiments, stability may instead be inferred from the primary held out performance without conducting additional resampling.

### VIII. Systems

In some instances, the systems may comprise e.g., one or more processors, and a memory unit communicatively coupled to the one or more processors and configured to store instructions that, when executed by the one or more processors, cause the system to perform any of the embodiments disclosed herein.

Similarly, non-transitory computer-readable storage media are disclosed that may comprise instructions for operating a system configured to perform any of the disclosed methods for sorting a potential HSCT donor as a donor or non-donor, for predicting a response to a mobilization agent, for training a machine learning model to predict the probability of an outcome following a HSCT or for training a machine learning model to predict donor response to a mobilization agent.

In some embodiments, the non-transitory computer-readable storage media storing one or more programs are described, the one or more programs comprising instructions, which when executed by one or more processors of a system, cause the system to perform any of the embodiments disclosed herein.

Provided herein are systems designed to implement any of the disclosed methods for sorting a potential HSCT donor as a donor or non-donor. The systems may comprise: one or more processors; a memory communicative coupled to the one or more processors and configured to store instructions that, when executed by the one or more processes, cause the system to: receive fluorescent intensity data generated by processing fluorescently-labeled sells from a sample from a potential HSCT donor using a flow cytometer; provide at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals; output a predicted probability of a positive first outcome following a HSCT for the potential HSCT donor; and sort the potential HSCT donor as a donor or a non-donor based at least on the predicted probability of a positive first outcome.

The systems may be configured to take in a user defined threshold that can be used for sorting a potential HSCT donor as a donor or non-donor. The exemplary system may comprise: one or more processors; an input device; a memory communicative coupled to the one or more processors and the input device and configured to store instructions that, when executed by the one or more processes, cause the system to: receive user defined threshold, receive fluorescent intensity data generated by processing fluorescently-labeled sells from a sample from a potential HSCT donor using a flow cytometer; provide at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals; output a predicted probability of a positive first outcome following a HSCT for the potential HSCT donor; and sort the potential HSCT donor as a donor or a non-donor based at least on the predicted probability of a positive first outcome, wherein the potential donor is sorted as a donor if the predicted probability from the first machine learning model is greater than the user defined threshold.

In some embodiments, the fluorescently-labeled cell have been labeled by fluorescently labeling cells contained withing the sample from the potential HSCT donor by contacting at least an aliquot of the sample with at least on immunophenotyping fluorescent labeling panel. In some embodiments, the system comprises a cell liquid handling device that can be configured to automatically fluorescently label cells contained within the sample from the potential HSCT donor by contacting a least an aliquot of the sample with at least on immunophenotyping fluorescent labeling panel.

Also provided herein are systems designed to train a machine learning model to predict the probability of an outcome, such as an outcome described herein, following a HSCT. The systems may comprise: one or more processors; a memory communicative coupled to the one or more processors and configured to store instructions that, when executed by the one or more processes, cause the system to: obtain fluorescent intensity data, generated from a plurality of fluorescently labeled cells from a donor of a plurality of donor individuals who have donated hematopoietic stem cells to a recipient individual of a plurality of recipient individuals; obtain one or more indications of the outcome of a matched recipient individual following an HSCT from a donor in the plurality of donor individuals; and train a machine learning model to predict the probability of an outcome following a HSCT, wherein the training is based at least on (a) a subset of the fluorescent intensity data, or data derived therefrom, for the plurality of donor individuals, and (b) one or more indications of the outcome of a matched recipient following an HSCT from the plurality of donor individuals.

Also provided herein are systems designed to train a machine learning model to predict donor response to a mobilization agent. The system may comprise: one or more processors; a memory communicative coupled to the one or more processors and configured to store instructions that, when executed by the one or more processes, cause the system to: obtain pre-mobilized fluorescent intensity data, generated from a plurality of fluorescently labeled cells from a pre-mobilized sample collected from a donor of a plurality of donor individuals; obtain post-mobilized fluorescent intensity data, generated from a plurality of fluorescently labeled cells from a post-mobilized sample collected from the donor; obtain one or more indications of the response of the donor to a mobilization agent from the post-mobilized fluorescent intensity data; and train a machine learning model to predict donor response to a mobilization agent, wherein the training is based at least on (a) a subset of the pre-mobilized fluorescent intensity data, or data derived therefrom, for the plurality of donor individuals, and (b) one or more indications of the response.

### A. Computer Processors & Systems

Provided herein are non-transitory computer-readable storage medium storing one or more programs, the one or more programs comprising instructions, which when executed by one or more processors of a system, cause the system to perform any of the methods described herein.

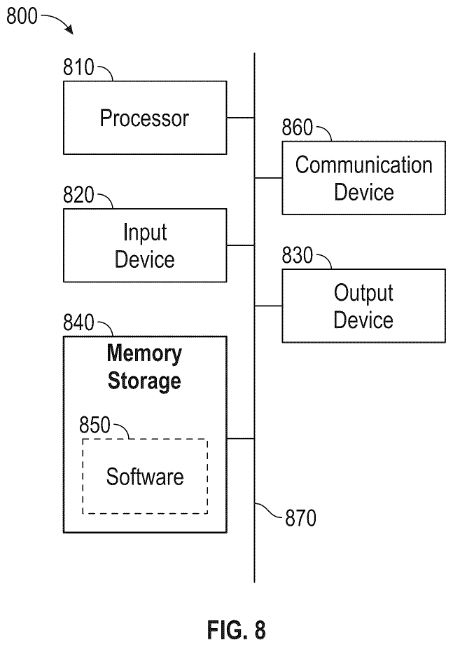

FIG. 8 illustrates an exemplary computing system, in accordance with some implementations. Computing system 800 can be a component of a system for sorting a potential HSCT donor as a donor or non-donor.

Computing system 800 can include a host computer connected to a network. Computing system 800 can be a client computer or a server. As shown in FIG. 8, computing system 800 can comprise any suitable type of microprocessor-based device, such as a personal computer; workstation; server; or handheld computing device, such as a phone or tablet. The computer can include, for example, one or more of processor 810, input device 820, output device 830, memory storage 840, and communication device 860.

Input device 820 can be any suitable device that provides input, such as a touch screen or monitor, keyboard, mouse, or voice-recognition device. Input device 820 can be configured to receive one or more use defined threshold. The user defined threshold may be any of the predetermined thresholds as described herein. Output device 830 can be any suitable device that provides output, such as a touch screen, monitor, printer, disk drive, or speaker.

Memory storage 840 can be any suitable device that provides storage, such as an electrical, magnetic, or optical memory, including a RAM, cache, hard drive, CD-ROM drive, tape drive, or removable storage disk. Communication device 860 can include any suitable device capable of transmitting and receiving signals over a network, such as a network interface chip or card. The components of the computer can be connected in any suitable manner, such as via a physical bus or wirelessly. Memory storage 840 can be a non-transitory computer-readable storage medium comprising one or more programs, which, when executed by one or more processors, such as processor 810, cause the one or more processors to execute any of the methods described herein.

Software 850, which can be stored in memory storage 840 and executed by processor 810, can include, for example, the programming that embodies the functionality of the present disclosure (e.g., as embodied in the methods, systems, computers, servers, and/or devices as described above). In some embodiments, software 850 can be implemented and executed on a combination of servers such as application servers and database servers.

Software 850 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include, but is not limited to, an electronic, magnetic, optical, electromagnetic, or infrared wired or wireless propagation medium.

Computing system 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 800 can implement any operating system suitable for operating on the network. Software 850 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### Exemplary Embodiments

Exemplary implementations of the methods and systems described herein include:
Embodiment 1. A method for sorting a potential Hematopoietic Stem Cell Transplantation (HSCT) donor as a donor or non-donor, comprising:
fluorescently labeling cells contained within a sample from a potential HSCT donor, by contacting at least an aliquot of the sample with at least one immunophenotyping fluorescent labeling panel,
generating fluorescent intensity data by processing the fluorescently-labeled cells from the sample using a flow cytometer;
providing at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals;
outputting a predicted probability of a positive first outcome following a HSCT for the potential HSCT donor; and
sorting the potential HSCT donor as a donor or a non-donor based at least on the predicted probability of a positive first outcome.
Embodiment 2. The method of embodiment 1, wherein the potential donor is sorted as a donor if the predicted probability from the first machine learning model is greater than a predetermined threshold.
Embodiment 3. The method of embodiment 1 or embodiment 2, further comprising generating a HSCT blood donation product from the potential HSCT donor if the potential HSCT donor is sorted as a donor.
Embodiment 4. The method of any of embodiments 1-3, further comprising treating the potential donor with a mobilization agent to mobilize hematopoietic stem cells from bone marrow to peripheral blood if the potential HSCT donor is a donor.
Embodiment 5. The method of embodiment 4, wherein the mobilization agent is granulocyte-colony stimulating factor (G-CSF).
Embodiment 6. The method of embodiment 1 or embodiment 2, further comprising matching the potential HSCT donor to a recipient in need of a HSCT donation if the potential HSCT donor is sorted as a donor.
Embodiment 7. The method of embodiment 6, further comprising transplanting cells from the potential HSCT donor to the recipient in need of a HSCT donation.
Embodiment 8. The method of any of embodiments 1-7, wherein the first machine learning model has been trained with results of a second machine learning model.
Embodiment 9. The method of embodiment 8, wherein the second machine learning model has been trained to predict the probability of a second outcome in a HSCT recipient with at least: (a) fluorescent intensity data, or data derived therefrom obtained from a plurality of fluorescently labeled cells from a plurality of HSCT donor individuals and (b) one or more indications of a second outcome for a plurality of recipient individuals who have each received a HSCT from an individual in the plurality of donor individuals.
Embodiment 10. The method of embodiment 8 or embodiment 9, wherein the second outcome is a clinical indication that partially explains the first outcome.
Embodiment 11. The method of embodiment 8 or embodiment 9, wherein the second outcome is a clinical indication that does not explain the first outcome.
Embodiment 12. The method of embodiment 8 or embodiment 9, wherein the second outcome is an indication of the positive first outcome collected at a second time point.
Embodiment 13. The method of any of embodiments 8-12, wherein the training the first machine learning model with a results of the second machine learning model improves predictive performance of the first machine learning model.
Embodiment 14. The method of embodiments 8-13, further comprising generating the results of a second machine learning model by providing at least a subset of the fluorescent intensity data, or data derived therefrom as input to the second machine learning model.
Embodiment 15. The method of embodiment 8, wherein the second machine learning model is a mobilization model.
Embodiment 16. The method of embodiment 15, wherein the mobilization model is a machine learning model trained to predict donor response to a mobilization agent.
Embodiment 17. The method of embodiment 16, wherein the mobilization model has been trained to predict donor response to a mobilization agent using at least (a) a subset of pre-mobilized fluorescent intensity data, or data derived therefrom, for a plurality of donor individuals, and (b) one or more indications of the response.
Embodiment 18. The method of embodiment 17, wherein the pre-mobilized fluorescent intensity data has been generated from a plurality of fluorescently labeled cells from a pre-mobilized sample collected from a donor of a plurality of donor individuals.
Embodiment 19. The method of embodiment 17 or embodiment 18, wherein the one or more indications of the response has been generated using post-mobilized fluorescent intensity data, generated from a plurality of fluorescently labeled cells from a post-mobilized sample collected from the donor.
Embodiment 20. The method of embodiment 1-7, wherein the first machine learning model has been trained with two or more indications of the positive first outcome collected at two or more timepoints.
Embodiment 21. The method of embodiment 20, wherein the predicted probability of a positive first outcome comprises the predicted probability of the positive first outcome at each of the two or more time points.
Embodiment 22. The method of embodiments 1-21, wherein the sorting is based on a predicted probability of a negative first outcome outputted from a third machine learning model.
Embodiment 23. The method of embodiment 22, wherein the third machine learning model has been trained to predict the probability of a negative first outcome following a HSCT with at least (a) fluorescent intensity data, or data derived therefrom obtained from a plurality of fluorescently labeled cells from a plurality of HSCT donor individuals and (b) one or more indications of a negative first outcome for a plurality of recipient individuals who have each received a HSCT from an individual in the plurality of donor individuals.
Embodiment 24. The method of embodiment 22 or embodiment 23, wherein the potential HSCT donor is sorted as a non-donor if the predicted probability of a negative first outcome following a HSCT is greater than a first predetermined threshold.
Embodiment 25. The method of any of embodiments 22-24, wherein the potential HSCT donor is sorted as a donor if the predicted probability of a negative first outcome following a HSCT is less than the first predeterminer threshold and the predicted probability of a positive first outcome following a HSCT is greater than a second predetermined threshold.
Embodiment 26. The method of any of embodiments 22-25, wherein additional clinical metrics are used to sort the potential HSCT donor as a donor or a non-donor if the predicted probability of a negative first outcome following a HSCT is less than the first predetermined threshold and the predicted probability of a positive first outcome following a HSCT is less than the second predetermined threshold.
Embodiment 27. The method of embodiment 23, wherein the third machine learning model has been trained with two or more indications of the negative first outcome collected at two or more timepoints.
Embodiment 28. The method of embodiment 27, wherein the predicted probability of a negative first outcome comprises the predicted probability of the negative first outcome at each of the two or more timepoints.
Embodiment 29. The method of any of embodiment 22-28, further comprising generating a predicted probability of a negative first outcome by providing at least a subset of the fluorescent intensity data, or data derived therefrom as input to the third machine learning model and outputting the predicted probability of a negative first outcome.
Embodiment 30. The method of any of embodiments 1-29, wherein the sample comprises peripheral blood cells.
Embodiment 31. The method of any of embodiments 1-30, wherein the sample comprises isolated peripheral blood mononuclear cells (PBMCs).
Embodiment 32. The method of any of embodiments 1-31, wherein the sample comprises a pre-mobilized sample.
Embodiment 33. The method of any of embodiments 1-14 and 20-31, wherein the sample comprises a post-mobilized sample.
Embodiment 34. The method of any of embodiments 1-33, wherein the potential HSCT donor is healthy.
Embodiment 35. The method of any of embodiments 1-34, wherein the positive first outcome is survival, lack of infection, lack of disease relapse, or lack of graft vs host disease (GvHD) between the HSCT and a defined timeframe.
Embodiment 36. The method of any of embodiments 9-35, wherein the second outcome is survival, lack of infection, lack of disease relapse, or lack of graft vs host disease (GvHD) between the HSCT and the defined timeframe.
Embodiment 37. The method of embodiment 35 or embodiment 36, wherein the negative first outcome is death when the positive first outcome is survival.
Embodiment 38. The method of embodiment 35 or embodiment 36, wherein the negative first outcome is infection when the positive first outcome is lack of infection.
Embodiment 39. The method of embodiment 35 or embodiment 36, wherein the negative first outcome is disease relapse when the positive first outcome is lack of disease relapse.
Embodiment 40. The method of embodiment 35 or embodiment 36, wherein the negative first outcome is GvHD when the positive first outcome is lack of GvHD.
Embodiment 41. The method of any of embodiments 35-40, wherein the defined timeframe is 6 months, 1 year, 2 years, 3 years, 4 years, or 5 years.
Embodiment 42. The method of any of embodiments 1-41, wherein the first machine learning model comprises a decision tree based classification model.
Embodiment 43. The method of any of embodiments 1-41, wherein the first machine learning model comprises a XGBoost model.
Embodiment 44. The method of any of embodiments 1-41, wherein the first machine learning model comprises a logistic regression model.
Embodiment 45. The method of any of embodiments 1-41, wherein the first machine learning model comprises a Adaptive Best Subset Selection Ensemble (ABSSE) model.
Embodiment 46. The method of any of embodiments 8-45, wherein the second machine learning model comprises a decision tree based classification model.
Embodiment 47. The method of any of embodiments 8-45, wherein the second machine learning model comprises a XGBoost model.
Embodiment 48. The method of any of embodiments 8-45, wherein the second machine learning model comprises a logistic regression model.
Embodiment 49. The method of any of embodiments 8-45, wherein the second machine learning model comprises a Adaptive Best Subset Selection Ensemble (ABSSE) model.
Embodiment 50. The method of any of embodiments 22-49, wherein the third machine learning model comprises a decision tree based classification model.
Embodiment 51. The method of any of embodiments 22-49, wherein the third machine learning model comprises a XGBoost model.
Embodiment 52. The method of any of embodiments 22-49, wherein the third machine learning model comprises a logistic regression model.
Embodiment 53. The method of any of embodiments 22-49, wherein the third machine learning model comprises a Adaptive Best Subset Selection Ensemble (ABSSE) model.
Embodiment 54. A method of training a machine learning model to predict the probability of an outcome following a HSCT, comprising:
obtaining fluorescent intensity data, generated from a plurality of fluorescently labeled cells from a donor of a plurality of donor individuals who have donated hematopoietic stem cells to a recipient individual of a plurality of recipient individuals;
obtaining one or more indications of the outcome of a matched recipient individual following an HSCT from a donor in the plurality of donor individuals; and
training a machine learning model to predict the probability of an outcome following a HSCT, wherein the training is based at least on (a) a subset of the fluorescent intensity data, or data derived therefrom, for the plurality of donor individuals, and (b) one or more indications of the outcome of a matched recipient following an HSCT from the plurality of donor individuals.
Embodiment 55. The method of embodiments 54, wherein the outcome is selected from a group consisting of survival, death, disease relapse, lack of disease relapse, infection, lack of infection, lack of HvGD, or HvGD.
Embodiment 56. The method of embodiment 54 or embodiment 55, wherein the machine learning model is further trained using two or more indication of the outcome collected at two or more timepoints.
Embodiment 57. The method of embodiment 56, wherein the predicted probability of an outcome comprises the predicted probability of the outcome at each of the two or more time points.
Embodiment 58. A method of training a machine learning model to predict donor response to mobilization agents, comprising:
obtaining pre-mobilized fluorescent intensity data, generated from a plurality of fluorescently labeled cells from a pre-mobilized sample collected from a donor of a plurality of donor individuals;
obtaining post-mobilized fluorescent intensity data, generated from a plurality of fluorescently labeled cells from a post-mobilized sample collected from the donor;
obtaining one or more indications of the response of the donor to a mobilization agent from the post-mobilized fluorescent intensity data; and
training a machine learning model to predict donor response to a mobilization agent, wherein the training is based at least on (a) a subset of the pre-mobilized fluorescent intensity data, or data derived therefrom, for the plurality of donor individuals, and (b) one or more indications of the response.
Embodiment 59. The method of embodiment 58, wherein the machine learning model is further trained using information about the cellular composition of the post-mobilized sample.
Embodiment 60. The method of embodiment 58 or embodiment 59, wherein the one or more indications of the response comprise number of CD34+ cells.
Embodiment 61. The method of any of embodiments 54-60, wherein the machine learning model comprises a decision tree based classification model.
Embodiment 52. The method of any of embodiments 54-60, wherein the machine learning model comprises a XGBoost model.
Embodiment 63. The method of embodiment 62, wherein training comprises optimizing performance using hyperparameter tuning.
Embodiment 64. The method of embodiment 63, wherein the hyperparameter tuning is performed using Grid Search.
Embodiment 65. The method of embodiment 63 or embodiments 64, wherein hyperparameters used for hyperparameter tuning comprise, number of trees, depth of trees, a learning rate, a fraction of training samples to build each tree, and a fraction of features used by each tree controlled by overfitting parameters.
Embodiment 66. The method of any of embodiments 54-60, wherein the machine learning model comprises a logistic regression model.
Embodiment 67. The method of any of embodiments 54-60, wherein the machine learning model comprises a Adaptive Best Subset Selection Ensemble (ABSSE) model.
Embodiment 68. The method of embodiment 67, wherein training the ABSSE model comprises multilayer feature selection.
Embodiment 69. The method of any of embodiments 54-68, wherein training the machine learning model comprises selecting two or more feature sets, training two or more machine learning model with the two or more feature sets.
Embodiment 70. The method of any of embodiments 54-69, wherein the fluorescent intensity data, generated from a plurality of fluorescently labeled cells is generated by a method comprising;
fluorescently labeling cells contained within a sample from the donor, by contacting at least an aliquot of the sample with at least one immunophenotyping fluorescent labeling panel;
generating fluorescent intensity data by processing the fluorescently-labeled cells from the sample using a flow cytometer.
Embodiment 71. The method of any one of embodiments 1-53, and 70, wherein one of the at least one immunophenotyping fluorescent labeling panel comprises a panel of fluorescent-labeled antibodies directed to cell surface proteins associated with antigen-presenting cells (APCs).
Embodiment 72. The method of embodiment 71, wherein the panel of fluorescently-labeled antibodies comprises fluorescently-labeled antibodies directed to CD3, CD4, CD8, CD25, CD45, CD19, CD27, IgD, IgM, CD56, CD16, CD14, HLA-DR, CD11c, CD56, TCRgd, TCR Vα7.2. TCR Vδ1, TCR Vδ2, TCR Vα24-Jα18, CCR10, CD103/ITGAE, CD122/IL2RB, CD161/KLRB1, CD223/LAG-3, CD274/PD-L1, CD335/NKp46, CD43, CD10, CD138, CD141, CD183/CXCR3, CD185/CXCR5, CD194/CCR4, CD197/CCR7, CD279/PD-1, CD28, CD294/CRTH2, CD337/NKp30, CD38, CD39, CD5, CD62L, CD86, CD95, ICOS, TIGIT, TIM-3, CD40, KLRG1, CD69, CD196/CCR6, CD1c, CD24, CD267/TACI, CD303/BDCA-2/CLEC4C, CD31, CD319, CD57, CD127, CD45RO, CD45RA, or any combination thereof.
Embodiment 73. The method of any one of embodiment 71 or embodiment 72, wherein the panel of fluorescently labeled antibodies comprise fluorescently-labeled antibodies directed to cell surface markers that are indicative of live cells, dead cells, or both.
Embodiment 74. The method of any one of embodiments 1-73, wherein the fluorescent intensity data, or data derived therefrom, comprises mean fluorescent intensity (MFI) data.
Embodiment 75. The method of any one of embodiments 1-74, wherein the fluorescent intensity data, or data derived therefrom, comprises cell classifications.
Embodiment 76. The method of claim 75, wherein the cell classifications are generated from fluorescent intensity data, or data derived therefrom, comprising mean fluorescent intensity (MFI) data.
Embodiment 77. The method of embodiment 75 or embodiment 76, wherein the cell classifications are generated by a cell classification machine learning model.
Embodiment 78. The method of any one of embodiments 75-77, wherein the cell classifications comprise cell ownership into at least 10, at least 20, at least 30, at least 40, at least 50, at least 60, at least 70, at least 80, at least 90, or at least 100 cell populations.
Embodiment 79. The method of embodiment 78, wherein the cell populations comprise distinct immune cell subpopulations.
Embodiment 80. The method of embodiment 79, wherein the distinct immune cell subpopulations comprise white blood cells (WBC), Eosinophils, Eosinophil/CD5+, Neutrophils, Neutrophils/big, Neutrophils/CD5+, Neutrophils/small. B-cells, B-cells/CD5-CD27−, Monocytes/CD56+, Monocytes/CD56−, NK-cells, Dendritic cells (DC), T-cells, iNKT cells, gamma delta T-cells (total GD), Vd1 cells, Vd2 cells, Vdx cells, Mucosal-associated invariant T (MAIT) cells, TEMRA cells, CD4 naïve cells, T helper cells, CD4 effector memory cells, Treg cells, Leukocytes, Helper T cells, Non-T/Non-NK B cells, Naïve T cells, Memory T cells, Naïve Cytotoxic T cells, Memory Cytotoxic T cells, Granulocytes, Activated T cells, Non-T/Non-B/Non-NK activated cells, or any combination thereof.
Embodiment 81. The method of any one of embodiments 1-53 and 68-80, wherein the flow cytometer is configured for at least about 5, at least about 10, at least about 15, at least about 20, at least about 30, at least about 40, at least about 50, at least about 60, at least about 70, at least about 80, at least about 90, or at least about 100 fluorescence detection channels.
Embodiment 82. The method of any one of embodiments 1-53 and 68-81, wherein the flow cytometer is a full spectrum flow cytometer.
Embodiment 83. The method of any one of embodiments 1-53 and 68-82, wherein the flow cytometer outputs cell classification.
Embodiment 84. The method of any one of embodiments 1-53 and 68-82, wherein the flow cytometer outputs mean fluorescent intensity (MFI) data.
Embodiment 85. A systems comprising:
one or more processors;
an input device;
a memory communicative coupled to the one or more processors and the input device and configured to store instructions that, when executed by the one or more processes, cause the system to:
receive user defined threshold, receive fluorescent intensity data generated by processing fluorescently-labeled sells from a sample from a potential HSCT donor using a flow cytometer;
provide at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals;
output a predicted probability of a positive first outcome following a HSCT for the potential HSCT donor; and
sort the potential HSCT donor as a donor or a non-donor based at least on the predicted probability of a positive first outcome, wherein the potential donor is sorted as a donor if the predicted probability from the first machine learning model is greater than the user defined threshold.
Embodiment 86. A system comprising:
one or more processors;
a memory communicative coupled to the one or more processors and configured to store instructions that, when executed by the one or more processes, cause the system to:
obtain fluorescent intensity data, generated from a plurality of fluorescently labeled cells from a donor of a plurality of donor individuals who have donated hematopoietic stem cells to a recipient individual of a plurality of recipient individuals;
obtain one or more indications of the outcome of a matched recipient individual following an HSCT from a donor in the plurality of donor individuals; and
train a machine learning model to predict the probability of an outcome following a HSCT, wherein the training is based at least on (a) a subset of the fluorescent intensity data, or data derived therefrom, for the plurality of donor individuals, and (b) one or more indications of the outcome of a matched recipient following an HSCT from the plurality of donor individuals.
Embodiment 87. A system comprising:
one or more processors;
a memory communicative coupled to the one or more processors and configured to store instructions that, when executed by the one or more processes, cause the system to:
obtain pre-mobilized fluorescent intensity data, generated from a plurality of fluorescently labeled cells from a pre-mobilized sample collected from a donor of a plurality of donor individuals;
obtain post-mobilized fluorescent intensity data, generated from a plurality of fluorescently labeled cells from a post-mobilized sample collected from the donor;
obtain one or more indications of the response of the donor to a mobilization agent from the post-mobilized fluorescent intensity data; and
train a machine learning model to predict donor response to a mobilization agent, wherein the training is based at least on (a) a subset of the pre-mobilized fluorescent intensity data, or data derived therefrom, for the plurality of donor individuals, and (b) one or more indications of the response.

### EXAMPLES

The following examples are included for illustrative purposes only and are not intended to limit the scope of the present disclosure.

### Example 1: Analysis of Blood Samples from Blood Donors

### Sample Collection and Preparation:

For this example, multiple modalities of patient samples containing immune cells can be used individually or in combination, including but not limited to whole blood samples or subsets of whole blood samples, such as peripheral blood mononuclear cells (PBMCs). These samples may be analyzed with or without cryopreservation.

Fresh Peripheral Blood: Peripheral blood samples are collected from healthy donors. The use of fresh blood samples is viable if samples are kept at ambient temperature (approximately 15° C.-25° C.) and processed within 54 hours; otherwise, samples can be cryopreserved.

Cryopreserved Peripheral Blood: Collected peripheral blood samples can be processed to isolate peripheral blood mononuclear cells (PBMCs), which are stored at −80° C. if processed within 1 week or stored in liquid or vapor phase nitrogen for longer-term storage. Additionally, fresh whole blood samples can be frozen by simply mixing with a basic cryopreservative without the need for PBMC isolation where the latter is not clinically viable. Frozen cell number, starting blood volume, and time from blood draw to preservation are essential metrics to record for downstream processing.

### Flow Cytometry Analysis

Full-spectrum flow cytometry is employed to analyze the immune cells from all sample types. A panel of markers to generate a comprehensive immune profile covering but not limited to:

a) Lineage markers for αβT cells, invariant T cells, γδT cells, B cells, NK cells, monocytes, macrophages, dendritic cells, neutrophils, eosinophils, and basophils. (CD3, CD4, CD8, CD25, CD45, CD19, CD27, IgD, IgM, CD56, CD16, CD14, HLA-DR, CD11c, CD56, TCRgd, TCR Vα7.2. TCR Vδ1, TCR Vδ2, TCR Vα24-Jα18)
b) Functional markers relating to but not limited to activation, migration, exhaustion senescence and memory status (CCR10, CD103/ITGAE, CD122/IL2RB, CD161/KLRB1, CD223/LAG-3, CD274/PD-L1, CD335/NKp46, CD43, CD10, CD138, CD141, CD183/CXCR3, CD185/CXCR5, CD194/CCR4, CD197/CCR7, CD279/PD-1, CD28, CD294/CRTH2, CD337/NKp30, CD38, CD39, CD5, CD62L, CD86, CD95, ICOS, TIGIT, TIM-3, CD40, KLRG1, CD69, CD196/CCR6, CD1c, CD24, CD267/TACI, CD303/BDCA-2/CLEC4C, CD31, CD319, CD57, CD127, CD45RO, CD45RA)

The final gating tree is constructed to visually represent the hierarchical relationship between the identified cell subsets. In this nonlimiting example, the following cell subsets are identified: CD45+ (Leukocytes), CD3+ (T cells), CD4+ (Helper T cells), CD8+ (Cytotoxic T cells), CD19+ (B cells), CD14+ (Monocytes), CD56+ (Natural Killer cells), CD16+ (Neutrophils), HLA-DR+ (Activated T cells), CD3-CD56+ (NK cells), CD3-CD19+ (Non-T/Non-NK B cells), CD3+CD16+ (NKT cells), CD4+CD45RA+ (Naïve T cells), CD4+CD45RO+ (Memory T cells), CD8+CD45RA+ (Naïve Cytotoxic T cells), CD8+CD45RO+ (Memory Cytotoxic T cells), CD14+HLA-DR+ (Activated Monocytes), CD16+CD45+ (Granulocytes), CD3-CD19-HLA-DR+ (Non-T/Non-B/Non-NK activated cells), CD3+HLA-DR+ (Activated T cells).

Each subset is defined by the sequential application of gates based on the specific markers identified in the staining panel.

The comprehensive immune profiles generated provide a high resolution overview of the immune shape of a donor. Signatures that are measured by this method can be but are not limited to single parameter increases or decreases as well as perturbations of the immune shape as dictated by changes in the combinatorial ratios of parameters.

### Example 2: Predicting Survival from Post-Mobilization Product

In this illustrative example using proxy data only, a machine learning model is developed to predict an outcome based on immune profile data according to the methods described herein. The machine learning model is developed to predict survival outcomes based on immune profile data sourced after a donor's haemopoietic stem cells (HSC) have been mobilized using granulocyte colony-stimulating factor (G-CSF) or a similar mobilizing agent. This process stimulates the release of stem cells from the bone marrow into the bloodstream, where they can be collected for transplantation. The immune profile data, comprising various immune cell populations and biomarkers, is then analyzed as described in Example 1 to determine its predictive value in assessing survival outcomes post-transplantation.

The dataset consists of a cohort of 771 samples, each representing an individual with associated immune parameters. These immune parameters, totaling 190 distinct features, capture various aspects of the immune system, such as cell populations and phenotypes. The goal of the model is to leverage these immune biomarkers to classify individuals into two groups: those who are likely to survive and those who are not, based on their immune profile. By training the model on historical data, the aim is to create a predictive tool that can assess an individual's survival probability by analyzing their immune characteristics, potentially assisting in risk stratification and treatment decision-making.

A random forest model is used to identify features from the immune parameters that are likely important for the survival outcome. FIG. 8 provides a graphical representations of the features selected by the RandomForest feature selection ranked by importance.

Next, a gradient boosting machine learning model is trained using all input data. The data representing input features and the target variable indicating survival status are used to train and evaluate a machine learning model. Of the 771 samples, 524 were coded as survived and 336 were coded as did not survive. The target variable initially consists of categorical values representing survival status, which are converted into numerical values where “NO” is replaced by 0 and “YES” is replaced by 1. This conversion is performed to enable numerical processing within the model.

The dataset is then split into training and testing sets, where 80% of the data is used for training the model, and the remaining 20% is reserved for testing. A fixed random state is applied to ensure the results are reproducible.

An XGBoost Classifier is instantiated with the following hyperparameters:
a) Objective: Set to binary: logistic for binary classification tasks.
b) n_estimators: Specifies that 300 decision trees will be built during training.
c) max_depth: Limits the maximum depth of each tree to 5, controlling the model's complexity.
d) learning_rate: Set to a small value of 0.01, which ensures the model learns gradually and helps prevent overfitting.
e) subsample: 80% of the training samples are used for each tree, reducing overfitting.
f) colsample_bytree: Each tree uses 80% of the available features, which also helps prevent overfitting.

The model is an implementation of the XGBoost (Extreme Gradient Boosting) algorithm, which is trained using the training data. Once the model is trained, it is used to predict the survival outcomes for the test set. The predicted values are generated both as binary classifications (either survival or non-survival) and as probabilities of survival for each test case.

To evaluate the performance of the trained model, two key metrics are calculated:
a) Accuracy: This metric represents the proportion of correctly predicted survival outcomes compared to the true outcomes in the test set.
b) ROC AUC (Receiver Operating Characteristic Area Under the Curve): This metric evaluates the model's ability to discriminate between the survival and non-survival classes, providing a measure of how well the model distinguishes between these outcomes.

The model performance is assessed by examining the accuracy and ROC AUC values, demonstrating how effectively the machine learning model predicts survival outcomes based on the provided input features.

Reviewing the inner trees of the XGBoost model reveals decision boundaries chosen during training to estimate the best possible reduction in error at each subsequent training step in the model. These trees are then assembled into a chain where the error from the first model is fed forward to the next and so on for 100 trees (in this embodiment).

The model's predictive performance was continuously monitored during training by evaluating the LogLoss metric after each tree was added to the ensemble. This allowed for a step-by-step assessment of how well the model was learning. After the first tree was added, the training LogLoss stood at 0.6656, indicating the initial fit of the model. As more trees were added, the LogLoss steadily decreased, reflecting the model's improved ability to reduce errors. After the second tree, the LogLoss dropped to 0.6406, and by the fifth tree, it had further decreased to 0.5851. This progressive reduction in LogLoss demonstrates the model's increasing effectiveness at fitting the data with each successive step.

Ultimately, the performance of this model architecture on the dataset resulted in an accuracy of 70.97% and a ROC AUC score of 0.7464. These metrics indicate the model's reasonable ability to correctly predict survival outcomes and discriminate between survival and non-survival cases.

### Hyperparameter Tuning

Hyperparameter tuning is employed to optimize the performance of the XGBoost machine learning model, which is designed to predict survival outcomes based on immune profile data. The goal of hyperparameter tuning is to adjust the model's key parameters to achieve the best predictive accuracy and generalizability to unseen data. In this case, the hyperparameters control aspects of how the XGBoost model learns from the data, such as the number of decision trees, the learning rate, and the depth of the trees.

To tune the hyperparameters, a process known as Grid Search is used. This method systematically tests multiple combinations of hyperparameters to identify the configuration that results in the best performance. Specifically, the XGBoost model is tuned by varying the following hyperparameters:
a) n_estimators: The number of decision trees to build. A higher value can improve model performance but also increases the risk of overfitting.
b) max_depth: The maximum depth of each decision tree. A deeper tree captures more complex relationships in the data but can lead to overfitting if set too high.
c) Learning_rate: Controls the rate at which the model learns. A smaller learning rate slows down the learning process, allowing for more gradual and stable convergence.
d) subsample: The fraction of training samples used to build each tree, helping prevent overfitting by introducing randomness into the training process.
e) colsample_bytree: The fraction of features used by each tree, further controlling overfitting by ensuring that not all features are considered for every split.

The grid search is performed with 5-fold cross-validation, meaning the dataset is split into five parts, and the model is trained and validated on different splits of the data to ensure robustness. The best hyperparameters are determined by evaluating the model's performance using the ROC AUC (Receiver Operating Characteristic Area Under the Curve) metric, which measures the model's ability to distinguish between survival and non-survival cases.

Once the grid search identifies the optimal combination of hyperparameters, the model is trained using these settings, and its performance is evaluated on a reserved test dataset. This process ensures that the XGBoost model achieves high accuracy and generalizability by finding the ideal balance between model complexity and performance.

In this example, 5 folds were fit for each of the 243 candidates, totaling 1215 model fits. The hyperparameter tuning process identified the optimal set of parameters for the XGBoost model. Cross-validation ensured that the model was evaluated on different subsets of the data, enhancing its robustness, and reducing the likelihood of overfitting. The best hyperparameters identified were: colsample_bytree set to 1.0, learning_rate set to 0.1, max_depth set to 7, n_estimators set to 300, and subsample set to 0.8. With these hyperparameters, the model achieved an accuracy of 72.26% and a ROC AUC score of 0.7882, reflecting an increase to its ability to differentiate between survival and non-survival outcomes.

### Example 3—Adding Clinical Metadata in Model (Relapse)

In this example, a machine learning model is developed using a two-step approach to predict a first outcome based on the probability of a second outcome as described herein. In this example, a machine learning model is developed using two-step approach to predict survival outcomes based on relapse probability and immune profile data collected after a donor's hematopoietic stem cells have been mobilized using granulocyte colony-stimulating factor (G-CSF) or a similar mobilizing agent. This process stimulates the release of stem cells from the bone marrow into the bloodstream, where they can be collected for transplantation. The immune profile data, comprising various immune cell populations and biomarkers, is then analyzed to determine its predictive value in assessing both relapse and survival outcomes post-transplantation.

The dataset consists of a cohort of 468 samples, each representing an individual with associated immune parameters and clinical measures including whether the individual experienced a relapse in their condition within one year. These immune parameters, totaling 380 features, represent sets of ratios of cell populations to either their immediate parent or to total white blood cells. This collection of ratios capture various aspects of the immune system, such as cell populations and phenotypes. In addition to the immune parameters, the dataset includes binary indicators of whether the patient experienced a relapse within one year following the transplant and survival at one year following the transplant. Of the 468 patients, 326 did not relapse within the first year, while 142 did. At the one-year mark, 274 patients survived, and 194 did not.

A machine learning model is developed to predict both relapse and survival outcomes based on immune profile data. The model uses two separate but connected steps to first predict relapse and then incorporate relapse probabilities into the prediction of survival outcomes.

### Dataset and Feature Engineering

The dataset consists of immune profile data which capture immune signatures such as various immune cell populations and biomarkers. This data set is joined with clinical response variables corresponding to overall survival at one year and onset of rejection within one year.

The final dataset contains the combined immune signature data, the binary relapse label, and the survival outcome.

### Mapping Binary Categorical Data

Two binary columns from the dataset are used for this analysis:
a) RELAPSE represents whether an individual has experienced a particular condition in the past three months.
b) SURVIVAL represents whether the individual has experienced certain life events related to health.

These columns are mapped to binary values of 0 and 1 for modeling. Any non-binary values are transformed into a binary format using mappings based on the unique values in the dataset.

### Preparing the Dataset

The immune signature data is merged with the binary labels for RELAPSE and SURVIVAL. This combined dataset is then split into training and test sets for both RELAPSE and SURVIVAL predictions. The initial split involves building a model to predict RELAPSE status based on the immune signature data, while SURVIVAL prediction is done in two stages: with and without incorporating predicted relapse probabilities.

### RELAPSE Prediction Using Logistic Regression

A Logistic Regression model is trained to predict the RELAPSE status. The configuration for the Logistic Regression model includes:
a) C=0.1: This is the inverse of the regularization strength. Smaller values indicate stronger regularization, which helps prevent overfitting.
b) penalty=‘l2’: This specifies L2 regularization (Ridge), which penalizes large coefficients, encouraging simpler models.
c) solver=‘lbfgs’: This is an optimization algorithm used for fitting the model, particularly suitable for smaller datasets and when L2 regularization is used.
d) random_state=9: This ensures that the results are reproducible by using the same random seed for data splitting and model initialization.

The features are scaled using MinMaxScaler, which transforms the features to a range between 0 and 1. The model is trained on the scaled training data and evaluated on the test data using ROC AUC as the performance metric.

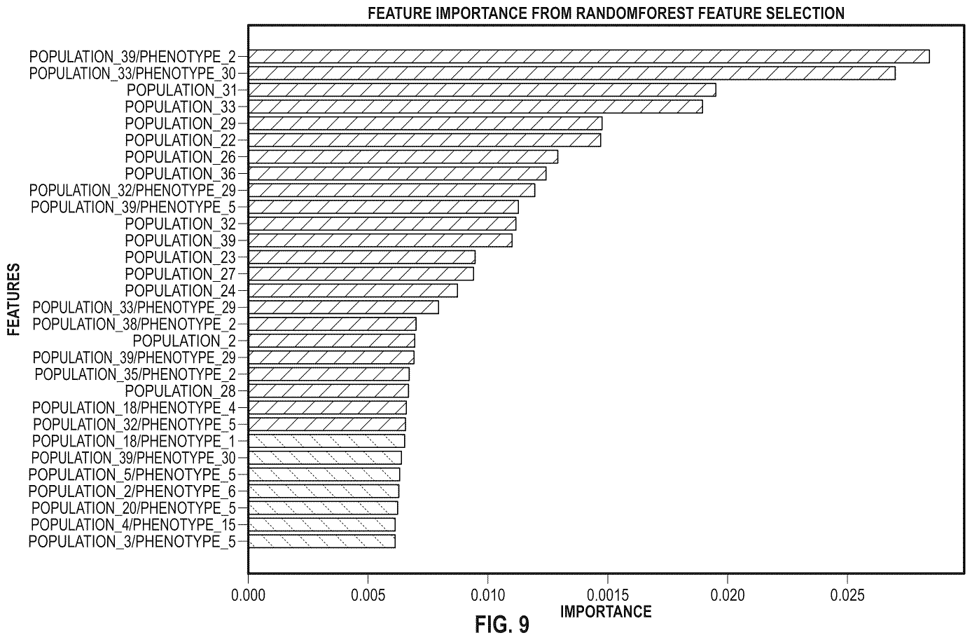

A random forest model is used to identify features from the immune parameters that are likely important for the disease relapse. FIG. 9 provides a graphical representations of the features selected by the RandomForest feature selection ranked by importance.

The ROC AUC for the RELAPSE prediction is 0.7013. This value indicates that the model performs moderately well in distinguishing between individuals who have experienced a relapse and those who have not.

SURVIVAL Prediction without Using RELAPSE Probabilities

A survival prediction model is trained without using the predicted relapse probabilities. The XGBoost (Extreme Gradient Boosting) model is used with the following configuration:
a) n_estimators=500: This specifies the number of boosting rounds (i.e., how many trees are grown in the model).
b) max_depth=7: The maximum depth of the trees is set to 7, controlling how complex each individual tree is. Deeper trees allow for more complex decision boundaries but can lead to overfitting.
c) learning_rate=0.01: This controls how much each tree contributes to the final prediction. A smaller learning rate makes the model learn more slowly but can improve generalization.
d) subsample=0.8: This ensures that only 80% of the training data is used to grow each tree, helping to prevent overfitting.
e) colsample_bytree=1: This specifies that all features are considered when splitting nodes in each tree.
f) random_state=9: This ensures reproducibility by controlling the randomness in the model training process.

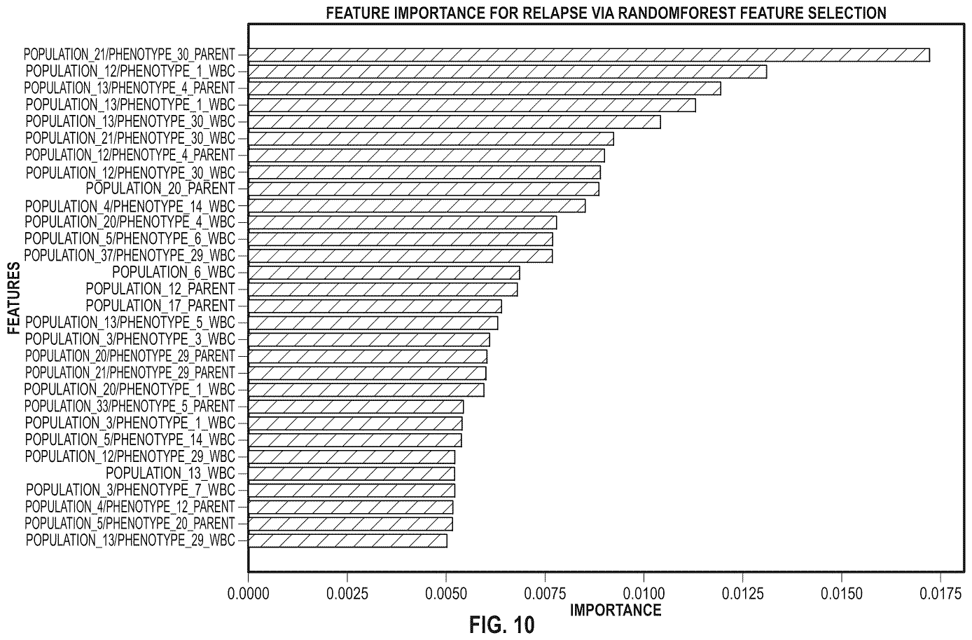

A random forest model is used to identify features from the immune parameters that are likely important for the SURVIVAL outcome. FIG. 10 provides a graphical representations of the features selected by the RandomForest feature selection ranked by importance.

The model is trained on the scaled survival data and evaluated using ROC AUC. The ROC AUC for SURVIVAL without RELAPSE is 0.6709. The initial survival model achieves a lower ROC AUC than the RELAPSE prediction model, indicating that it has more difficulty predicting the outcome without additional information.

Incorporating RELAPSE Probabilities into the SURVIVAL Model

In this step, the predicted RELAPSE probabilities are added as a new feature to the survival data. The RELAPSE probabilities are scaled using MinMaxScaler to ensure they are on the same scale as the other features. The model configuration remains the same as in the previous step.

The XGBoost model is then retrained, this time using both the immune signature features and the predicted relapse probabilities as inputs.

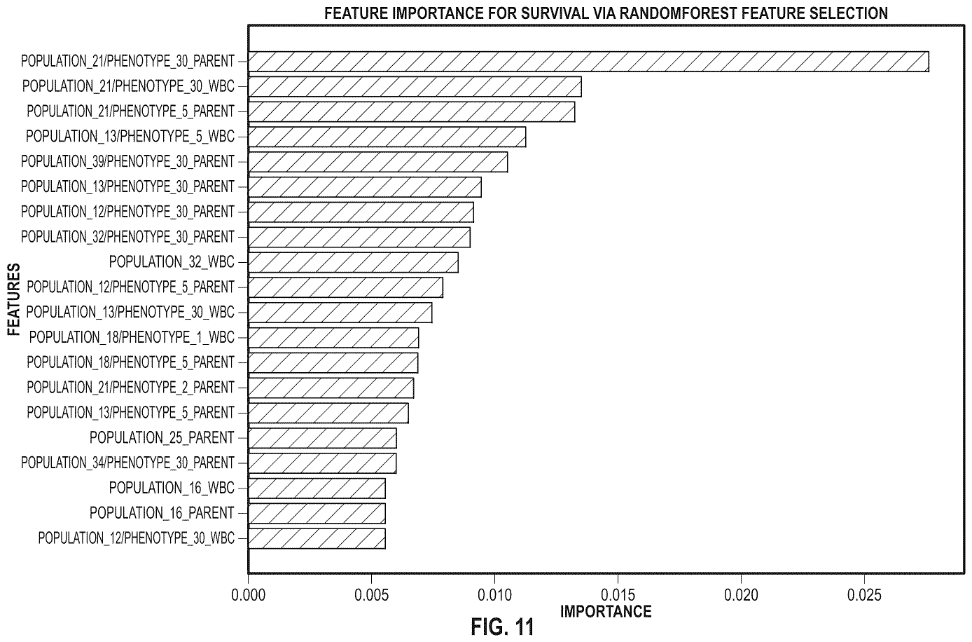

A random forest model is used to identify features for the SURVIVAL with RELAPSE model. FIG. 11 provides a graphical representations of the features selected by the RandomForest feature selection ranked by importance.

The model is evaluated again using ROC AUC. The ROC AUC for the SURVIVAL with RELAPSE models is 0.7184. Incorporating the relapse probabilities improves the performance of the survival prediction model.

The difference in ROC AUC between the survival model with and without relapse probability is calculated. The improvement in ROC AUC with relapse is 0.0476. This positive improvement indicates that incorporating predicted relapse probabilities enhances the survival model's performance.

The models used in this example—Logistic Regression for relapse and XGBoost for survival—are illustrative and can be swapped for other algorithms as described herein, such as Random Forest, Support Vector Machines, or Neural Networks. Similarly, the hyperparameters (e.g., learning rate, regularization strength, number of trees) can be tuned or adjusted to meet specific modeling requirements. This flexibility allows for customization depending on the dataset and the problem being addressed. Together, the results show the inclusion of predicted relapse probabilities improved the survival model's predictive power, showcasing the utility of multi-step modeling in this scenario. However, the models and configurations can be easily adapted to suit different use cases.

### Example 4—Dual Modeling

This example demonstrates how dual modeling of a positive and negative outcome according to embodiments described herein can be used to improve prediction. In this illustrative example, both survival (positive first outcome) and mortality (negative first outcome) outcomes are predicted based on immune profile data derived from the hematopoietic stem cell transplantation (HSCT) material that has been implanted in patients. The immune parameters, represented as ratios of immune cell populations, serve as predictors of post-transplant outcomes. Two separate ML models are employed-one to predict survival and the other to predict mortality. Their binary outputs are combined to assess the overall outcome prediction of the HSCT material.

### Input Data and Immune Parameters

The dataset consists of immune parameters extracted from the implanted HSCT material, capturing the composition and functionality of the immune system. These parameters are expressed as ratios of immune cell populations to either their immediate parent populations or total white blood cells, providing insights into the potential for immune reconstitution post-transplant.

In this analysis, the dataset contains 860 patients with binary outcomes for both survival and mortality, as indicated by the following breakdown:
a) 524 patients survived and did not die (Survive=1, Die=0).
b) 336 patients did not survive and died within one year post-transplant (Survive=0, Die=1)

The input data set includes:
a) Immune parameters: 380 features representing immune cell population ratios.
b) Binary clinical outcomes: Survival (YES/NO) and mortality (YES/NO) at one year post-transplant.

### Dual XGBoost Models for Survival and Mortality Prediction

A first model is developed to predict survival (Model 1). The objective of Model 1 is To predict the probability that a patient will survive one year after transplantation based on the immune profile of the implanted HSCT material. The model takes in immune parameters derived from the HSCT material. The output is a binary classification indicating whether the patient is likely to survive at one year (Survive=YES/NO).

Model 1 is configured:
a) n_estimators=500: Number of trees in the XGBoost ensemble.
b) max_depth=7: Maximum depth of each tree, controlling model complexity.
c) learning_rate=0.01: Learning rate, which controls the contribution of each tree.
d) subsample=0.8: Fraction of the training data used for growing each tree.
e) colsample_bytree=0.8: Fraction of features used for building each tree.
f) random_state=42: Ensures reproducibility of the results.

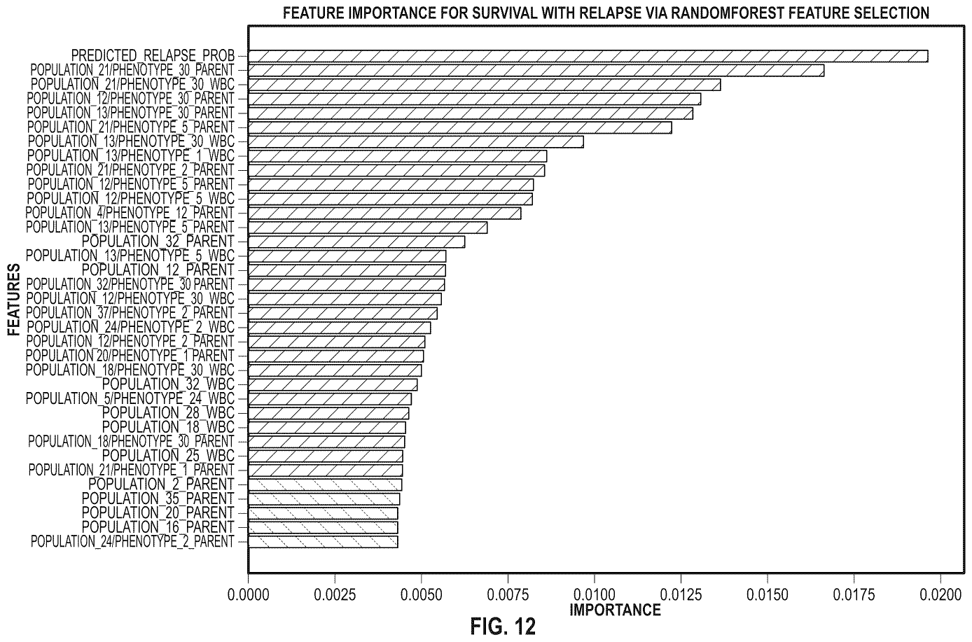

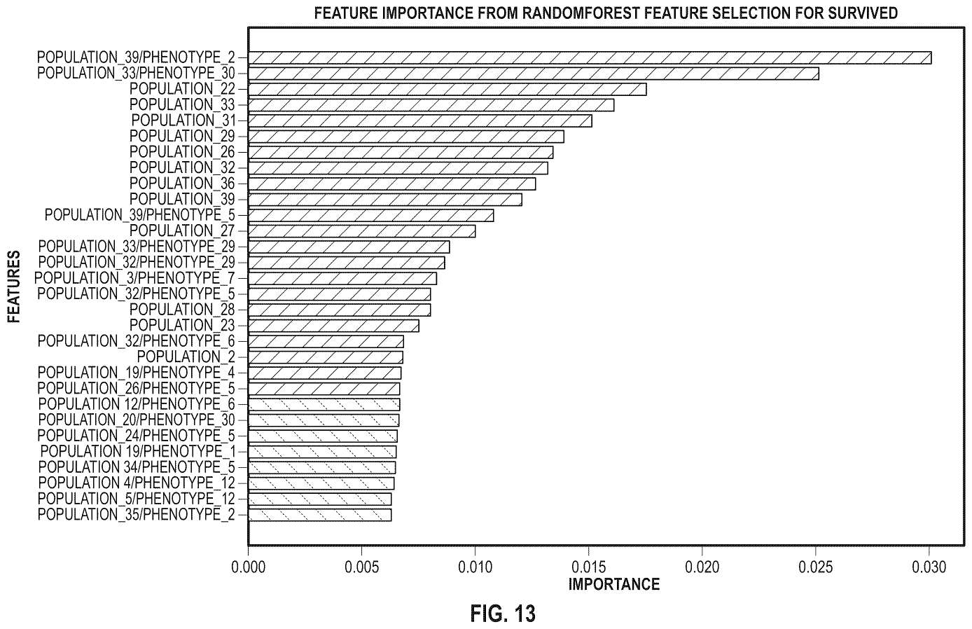

A random forest model is used to identify features from the immune parameters that are likely important for the SURVIVED model. FIG. 12 provides a graphical representations of the features selected by the RandomForest feature selection ranked by importance.

A second model is developed to predict mortality (Model 2.) The objective of Model 2 is to predict the probability that a patient will die within one year post-transplantation based on the same set of immune parameters. The input for Model 2 is immune parameters derived from the HSCT material. The output is a binary classification indicating whether the patient is likely to die by one year (Die=YES/NO). Model 2 is configured using the same XGBoost model configuration as used for Model 1.

Both XGBoost models are trained using the same set of immune parameters, with different target labels:
a) Model 1 (Survive): Trained to predict the binary survival outcome (YES/NO) at one year post-transplant.
b) Model 2 (Die): Trained to predict the binary mortality outcome (YES/NO) at one year post-transplant.

Both models use binary cross-entropy as the loss function, and their performance is evaluated using metrics such as ROC AUC (area under the receiver operating characteristic curve) and accuracy to assess the model's ability to correctly classify survival and mortality outcomes.

### Combining Predictions for Final Assessment

After training both XGBoost models, their binary predictions (Survive=YES/NO, Die=YES/NO) are combined to generate a final classification for assessing the quality of the HSCT material. Table 2 provides an exemplary classification contingency table based on the results of both models.

TABLE 2
Exemplary contingency model for sorting donors
based on Survival and Mortality models

| | Survive = YES | Survive = NO |
|---|---|---|
| Die = | Indicates uncertainty, suggesting the | Poorly Indicated HSCT Material: This |
| YES | need for further clinical assessment. | outcome indicates a low probability of |
| | | survival and a high probability of death, |
| | | suggesting that the HSCT material is |
| | | unfavorable. |
| Die = | Optimal HSCT Material: This | Indicates uncertainty, suggesting the |
| NO | outcome indicates a high probability | need for further clinical assessment. |
| | of survival and a low probability of | |
| | death, suggesting that the HSCT | |
| | material is favorable. | |

For the validation set, consisting of 154 patients: 108 patients were classified as optimal outcomes (Survive=YES, Die=NO), meaning they had a high probability of survival and a low probability of mortality, indicating favorable HSCT material.

This dual-model approach leverages immune data from the transplanted HSCT material to predict both survival and mortality outcomes. By combining the binary predictions from each model, clinicians can assess the quality of the HSCT material and its likelihood of leading to positive patient outcomes. The use of XGBoost, a powerful gradient-boosting algorithm, provides robust predictive modeling for this clinically significant task.

### Example 5—Prediction of Donors with Good Responses to Mobilization Expansion

Using the immune profiling platform as a robust method for predicting donor response to mobilization agents through paired blood sample analysis is also characterized here. Blood samples are processed using the immune profiling pipeline described in Example 1, including analysis of both pre-mobilization and post-mobilization measurements of key cell populations.

A machine learning model is then trained to predict donor responsiveness using as input the pre-mobilization ratios, and predicting response to mobilizing agent as compared to post-mobilized immune profile. The predictions from this model will indicate significant increases in relevant cell populations. This prediction provides a key indicator of the donor's likely outcome in response to the mobilization agent. The process can be further extended by chaining the prediction of post-mobilization outcomes to downstream effects, creating a robust model for identifying high-potential donors.

An additional method exists to include both pre- and post-mobilization data as input to an ML model that predicts patient outcomes, providing valuable guidance for donor selection and treatment strategies.

### Example 6—Predicting Relapse from Pre-Mobilization Product

The methods described in Example 1 were used to generate immune profiles for 145 healthy donors ahead of stem cell mobilization. Post-mobilization apheresis material from these donors was subsequently transplanted into 145 unrelated recipients in remission from acute myeloid leukemia (AML). 75% of cases were matched (8/8 HLA match) and the remaining were mismatched (6/8 or 7/8 HLA match). 75% of patients were transplanted during their first complete remission from AML, and ˜45% had an adverse European Leukemia Network risk profile. The Adaptive Best Subset Selection Ensemble (ABSSE) approach was employed to produce a model predictive of patient clinical relapse outcome from the donor immune profile.

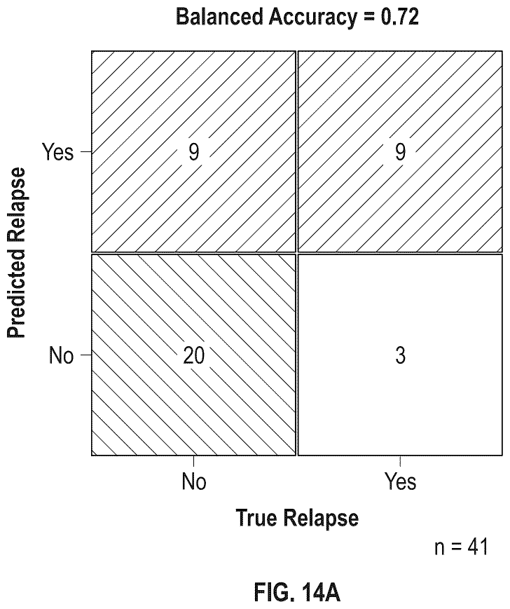

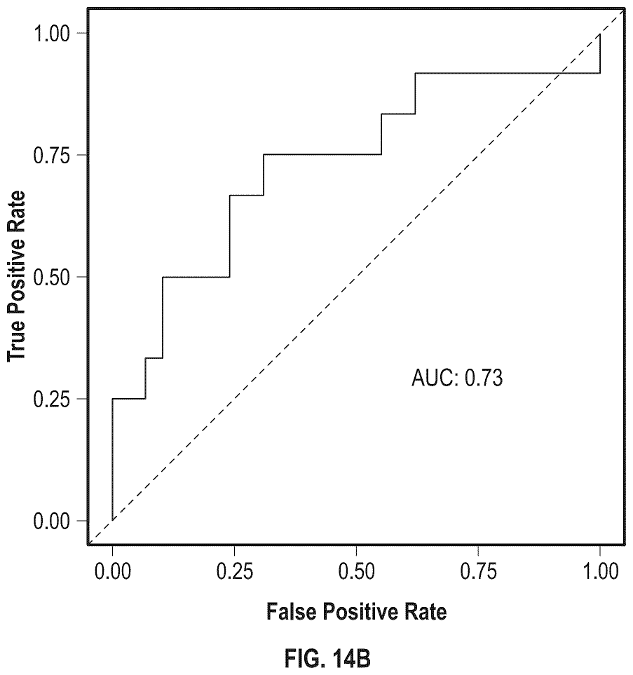

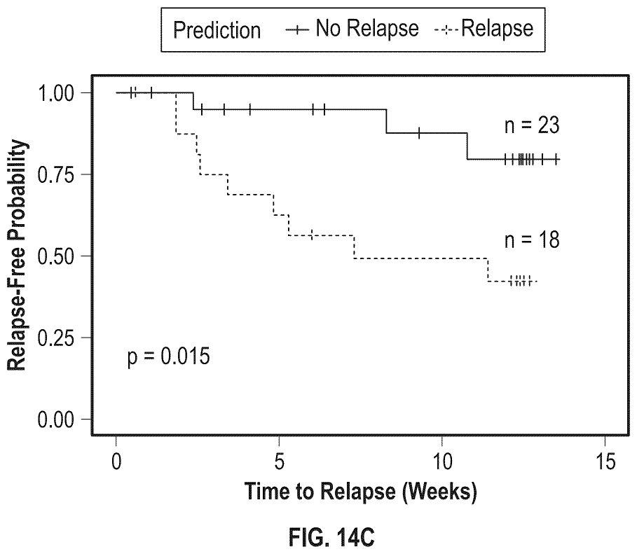

An ABSSE model was trained on 104 donor profiles and patient relapse outcomes as recorded in the ˜24 week post-transplant period. The performance of the model was assessed on a blind validation set of 41 donor/patient pairs. The model achieved promising performance of AUC=0.73 (FIG. 14A), successfully classifying relapse cases from donor immune profiles with a balanced accuracy of 0.72 (FIG. 14B), regardless of patient risk factors or donor/patient demographics.

Relapse-free probability was estimated in patients transplanted with donor material predicted to lead to relapse or predicted not to lead to relapse. The relapse-free probability was calculated using the Kaplan-Meier nonparametric survival analysis method to estimate the probability of relapse across the group of patients at a given time. This is an estimation, because every patient was only measured once at one given time (i.e. they are censored), and after that they were presumed to remain in the same status. Relapse-free probability allowed for estimation of the fraction of patients in each group remaining relapse-free at each time point.

Relapse-free probability was recalculated upon every incidence of relapse (i.e. every downward step); events where patients were measured and remain non-relapsing are marked by a tick in FIG. 14C. As shown in FIG. 14C, patients transplanted with donor material predicted to lead to relapse showed consistently worse relapse-free probability over the 12 week post-transplant period than predicted non-relapse cases, p=0.015 by log rank test (FIG. 14C).

These results demonstrate the feasibility of predicting the likelihood of AML relapse in patients, regardless of their own risk factors, based upon the immune profile of the unrelated donor. In additions, these data suggest that application of this method to select donors less likely to lead to relapse of the transplant recipient could reduce the relapse rate of AML post-allogeneic transplantation by ˜56%, a potentially profound improvement on the outcome of this widespread and important therapeutic intervention.

1. A method for sorting a potential Hematopoietic Stem Cell Transplantation (HSCT) donor as a donor or non-donor, comprising:
fluorescently labeling cells contained within a sample from a potential HSCT donor, by contacting at least an aliquot of the sample with at least one immunophenotyping fluorescent labeling panel,
generating fluorescent intensity data by processing the fluorescently-labeled cells from the sample using a flow cytometer;
providing at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals;
outputting a predicted probability of a positive first outcome following a HSCT for the potential HSCT donor; and
sorting the potential HSCT donor as a donor or a non-donor based at least on the predicted probability of a positive first outcome.

2. The method of claim 1, wherein the potential donor is sorted as a donor if the predicted probability from the first machine learning model is greater than a predetermined threshold.

3. The method of claim 1, further comprising generating a HSCT blood donation product from the potential HSCT donor if the potential HSCT donor is sorted as a donor.

4. The method of claim 1, further comprising treating the potential donor with a mobilization agent to mobilize hematopoietic stem cells from bone marrow to peripheral blood if the potential HSCT donor is a donor.

5. The method of claim 1, further comprising transplanting cells from the potential HSCT donor to a recipient in need of a HSCT donation, if the potential HSCT donor is sorted as a donor.

6. The method of claim 1, wherein the first machine learning model has been trained with results of a second machine learning model, wherein the second machine learning model has been trained to predict the probability of a second outcome in a HSCT recipient with at least: (a) fluorescent intensity data, or data derived therefrom obtained from a plurality of fluorescently labeled cells from a plurality of HSCT donor individuals and (b) one or more indications of a second outcome for a plurality of recipient individuals who have each received a HSCT from an individual in the plurality of donor individuals.

7. The method of claim 6, wherein the second machine learning model is a mobilization model, wherein the mobilization model is a machine learning model trained to predict donor response to a mobilization agent.

8. The method of claim 7, wherein the mobilization model has been trained to predict donor response to a mobilization agent using at least (a) a subset of pre-mobilized fluorescent intensity data, or data derived therefrom, for a plurality of donor individuals, and (b) one or more indications of the response.

9. The method of claim 1, wherein the first machine learning model has been trained with two or more indications of the positive first outcome collected at two or more timepoints, wherein the predicted probability of a positive first outcome comprises the predicted probability of the positive first outcome at each of the two or more time points.

10. The method of claim 1, wherein the sorting is based on a predicted probability of a negative first outcome outputted from a third machine learning model, wherein the third machine learning model has been trained to predict the probability of a negative first outcome following a HSCT with at least (a) fluorescent intensity data, or data derived therefrom obtained from a plurality of fluorescently labeled cells from a plurality of HSCT donor individuals and (b) one or more indications of a negative first outcome for a plurality of recipient individuals who have each received a HSCT from an individual in the plurality of donor individuals.

11. The method of claim 10, further comprising generating a predicted probability of a negative first outcome by providing at least a subset of the fluorescent intensity data, or data derived therefrom as input to the third machine learning model and outputting the predicted probability of a negative first outcome.

12. The method of claim 1, wherein the sample comprises peripheral blood cells or isolated peripheral blood mononuclear cells (PBMCs).

13. The method of claim 1, wherein the positive first outcome is survival, lack of infection, lack of disease relapse, or lack of graft vs host disease (GvHD) between the HSCT and a defined timeframe.

14. The method of claim 1, wherein the first machine learning model comprises a decision tree based classification model, a XGBoost model, logistic regression model or an Adaptive Best Subset Selection Ensemble (ABSSE) model.

15. The method of claim 1, wherein one of the at least one immunophenotyping fluorescent labeling panel comprises a panel of fluorescent-labeled antibodies directed to cell surface proteins associated with antigen-presenting cells (APCs).

16. The method of claim 1, wherein the fluorescent intensity data, or data derived therefrom, comprises mean fluorescent intensity (MFI) data and/or cell classifications.

17. The method of claim 16, wherein the cell classifications are generated from fluorescent intensity data, or data derived therefrom, comprising mean fluorescent intensity (MFI) data.

18. A method of training a machine learning model to predict the probability of an outcome following a HSCT, comprising:
obtaining fluorescent intensity data, generated from a plurality of fluorescently labeled cells from a donor of a plurality of donor individuals who have donated hematopoietic stem cells to a recipient individual of a plurality of recipient individuals;
obtaining one or more indications of the outcome of a matched recipient individual following an HSCT from a donor in the plurality of donor individuals; and
training a machine learning model to predict the probability of an outcome following a HSCT, wherein the training is based at least on (a) a subset of the fluorescent intensity data, or data derived therefrom, for the plurality of donor individuals, and (b) one or more indications of the outcome of a matched recipient following an HSCT from the plurality of donor individuals.

19. The method of claim 18, wherein the machine learning model comprises a decision tree based classification model, a XGBoost model, a logistic regression model and/or an Adaptive Best Subset Selection Ensemble (ABSSE) model.

20. A systems comprising:
one or more processors;
an input device;
a memory communicative coupled to the one or more processors and the input device and configured to store instructions that, when executed by the one or more processes, cause the system to:
receive user defined threshold, receive fluorescent intensity data generated by processing fluorescently-labeled sells from a sample from a potential HSCT donor using a flow cytometer;
provide at least a subset of the fluorescent intensity data, or data derived therefrom as input to a first machine learning model, wherein the first machine learning model has been trained to predict the probability of a positive first outcome following a HSCT with at least: (a) fluorescent intensity data, or data derived therefrom obtained from a plurality of fluorescently labeled cells from a plurality of HSCT donor individuals and (b) one or more indications of a positive first outcome for a plurality of recipient individuals who have each received a HSCT from an individual in the plurality of donor individuals;
output a predicted probability of a positive first outcome following a HSCT for the potential HSCT donor; and
sort the potential HSCT donor as a donor or a non-donor based at least on the predicted probability of a positive first outcome, wherein the potential donor is sorted as a donor if the predicted probability from the first machine learning model is greater than the user defined threshold.
